(19) United States
(12) Patent Application Publication
(10) Pub. No.: US 2004/0133115 A1
(43) Pub. Date: Jul. 8, 2004

(54) Method and apparatus for identifying heart rate feature events

(76) Inventors: Emily F. Hamilton, Verdun (CA); Michael C. Glaude, Anjou (CA); Maciej Macieszczak, Kanata (CA); Philip A. Warrick, Montreal (CA)

Correspondence Address: KENYON & KENYON, ONE BROADWAY, NEW YORK, NY, 10004, US

(21) Appl. No.: 10/698,232

(22) Filed: Oct. 31, 2003

Related U.S. Application Data
(60) Provisional application No. 60/422,893, filed on Nov. 1, 2002.

(51) Int. Cl. A61B 5/0444

(52) U.S. Cl. 600/511

(57) ABSTRACT

A method and apparatus for segmenting a heart rate signal to identify heart rate feature events are provided. A heart rate signal including a sequence of sample points is received. The heart rate signal is processed to generate a set of segments. Each segment is formed by enclosing a portion of the heart rate signal in a respective bounded area commencing at a start sample point and terminating at an end sample point of the heart rate signal. The sample points between the start sample point and end sample point lie within the bounded area. The set of segments are then processed to generate a plurality of sections, each section being indicative of a heart rate feature. The heart rate feature is selected from the set consisting of an acceleration event, a deceleration event and a baseline event. A signal indicative of the plurality of sections is then released.

### FIELD OF THE INVENTION

The present invention relates generally to the field of electronic heart rate monitoring and, more particularly, to a method and apparatus for identifying heart rate features in a heart rate signal such as regions of baseline, acceleration and deceleration. The method is particularly applicable to fetal heart rate monitoring systems.

### BACKGROUND OF THE INVENTION

A commonly used method for evaluating patient well-being relies on the analysis of the patient's heart rate using electronic heart rate monitors. These monitors measure the heart rate of the patient and generally produce a paper print out of the tracing over a period of time. Alternatively, the tracings over the most recent period of time are displayed on video screen displays. In the case where the patient is a fetus in-utero, an electronic fetal heart rate monitor is used. These monitors measure both the fetal heart rate and the mother's uterine contraction pattern and provide a reading of these measurements either in the form of a paper print out or a display on a display screen. The clinical staff is then able to use visual methods to study the tracings and deduce the degree of patient well-being. Abnormal patterns can lead to interventions such as more diagnostic tests, drug treatment or surgical intervention. The features of the heart rate that are used by clinicians to deduce patient well-being include baseline, acceleration, deceleration and heart rate variability.

A deficiency with the above-described method is that it does not enable an objective quantification of multiple features of the tracing, since the analysis is done visually by a doctor or nurse. Therefore, the analysis is subject to imprecision and normal human biases. Physicians show great variation in how they measure, label and interpret heart rate patterns, particularly when the patterns are measured only by visual inspection of the paper recording. While doctors and nurses are trained and presumably competent in their ability to assess the strip, there can be differences of opinion that may result in different clinical interventions.

In the context of the above, there is a need in the industry to provide a method and device for providing heart rate feature analysis that alleviates at least in part the problems associated with the existing methods.

### SUMMARY OF THE INVENTION

In accordance with a first broad aspect, the invention provides a method for segmenting a heart rate signal to identify heart rate feature events. The method comprises receiving a heart rate signal including a sequence of sample points, processing the heart rate signal to generate a set of segments, each segment being formed by enclosing a portion of the heart rate signal in a respective bounded area. The bounded area commences at a start sample point of the heart rate signal and terminates at an end sample point of the heart rate signal. The sample points between the start sample point and end sample point lie within the bounded area. The set of segments is then processed to generate a plurality of sections. A signal indicative of the plurality of sections is then released.

In accordance with a specific implementation, the heart rate feature is selected from the set consisting of an acceleration event, a deceleration event and a baseline event.

In accordance with another broad aspect, the invention provides an apparatus for implementing the above-described method.

In accordance with yet another broad aspect, the invention provides a computer readable medium including a program element suitable for execution by a computing apparatus for performing the above-described method.

In accordance with another broad aspect, the invention provides a system that comprises a sensor for receiving a signal indicative of a fetal heart rate and an apparatus suitable for monitoring the condition of a fetus. The apparatus comprises an input coupled to the sensor for receiving a signal indicative of a fetal heart rate, a feature detection module coupled to the input. The feature detection module implements a first processing unit adapted for processing the heart rate signal to generate a set of segments. Each segment is generated by enclosing a portion of the heart rate signal in a respective bounded area, the bounded area commencing at a start sample point of the heart rate signal and terminating at an end sample point of the heart rate signal. The sample points between the start sample point and the end sample point lie within the bounded area. The feature detection module also includes a second processing unit adapted for processing the set of segments to generate a plurality of sections, each section being indicative of a heart rate feature. The apparatus also includes a post processing module coupled to the feature detection module, the post processing module being adapted for deriving information on the basis of the heart rate features associated with the set of segments. The apparatus further comprises an output for releasing the information derived from the heart rate features associated with the set of segments. The system further comprising an output unit coupled to the output of the apparatus. The output unit is suitable for displaying the information derived from the heart rate features associated with the set of segments.

These and other aspects and features of the present invention will now become apparent to those of ordinary skill in the art upon review of the following description of specific embodiments of the invention in conjunction with the accompanying drawings.

### BRIEF DESCRIPTION OF THE DRAWINGS

In the accompanying drawings:

FIG. 1 shows a high level block diagram of a fetal heart rate monitoring system in accordance with a specific example of implementation of the present invention;

FIG. 2 shows a block diagram of a processing unit operative to generate information data derived from a heart rate signal, in accordance with a specific example of implementation of the present invention;

FIG. 3 shows a block diagram of a feature detection module operative to generate a list of labelled heart rate sections, the feature detection module including a bounded segment set generator 300 and a feature identification unit 302 in accordance with a specific example of implementation of the present invention;

FIG. 4a illustrates in simplified form a portion 450 of a heart rate signal 458 enclosed within a bounded area 452 in accordance with a specific non-limiting example of implementation of the invention;

FIG. 4b shows a flow diagram of a process implemented by the bounded segment set generator 300 of the feature extraction module of FIG. 3 for generating a set of bounded segments in accordance with a specific example of implementation of the present invention;

FIGS. 5a and 5b show in simplified form a signal diagram of a heart rate signal where a segment has been formed in accordance with a specific example of implementation of the present invention;

FIG. 6 shows a functional block diagram of the bounded segment set generator 300 in accordance with a specific example of implementation of the present invention;

FIG. 7 shows a block diagram of a segment search unit operative to generate a list of heart rate segments in accordance with a specific example of implementation of the present invention;

FIGS. 8a and 8b show in simplified form a signal diagram of a heart rate signal where a segment has been formed in accordance with a specific example of implementation of the present invention;

FIG. 9a is a signal diagram depicting a portion of a fetal heart rate signal and associated variability measure;

FIG. 9b shows in simplified form a signal diagram depicting variability information in accordance with a specific example of implementation of the present invention;

FIG. 10 shows a graph of a function for determining a maximum allowable drift for a bounded area having a certain length in accordance with a specific example of implementation of the present invention;

FIG. 11 shows in simplified form a signal diagram of a heart rate signal having a plurality of extrema bounded areas and extrema regions in accordance with a specific example of implementation of the present invention;

FIG. 12 shows in simplified form a signal diagram of a heart rate signal having a plurality of points for determining a longest bounded area of the heart signal in accordance with a specific example of implementation of the present invention;

FIG. 13 shows in simplified form a signal diagram of a heart rate signal having a portion enclosed within a longest bounded heart rate region and a polynomial-fitted line shown in dotted lines in accordance with a specific example of implementation of the present invention;

FIGS. 14A and 14B show a representation of a method of determining a longest polynomial bounded area of a heart rate signal in accordance with a specific example of implementation of the present invention;

FIG. 15 shows a block diagram of a feature identification unit operative to generate a list of labelled heart rate sections in accordance with a specific example of implementation of the present invention;

FIG. 16 shows in simplified form a signal diagram of a heart rate signal having two segments separated in time by a duration Δt;

FIG. 17 shows in simplified form a signal diagram of a heart rate signal having segments that are separated by a short sharp deviation in accordance with a specific example of implementation of the present invention;

FIG. 18 shows in simplified form two signal diagrams of a heart rate signal having segments that are classified as either baseline segments or non-baseline segments in accordance with a specific example of implementation of the present invention;

FIG. 19 shows in simplified form a fetal heart rate signal with a plurality of segments with their line of best fit in accordance with a specific example of implementation of the present invention;

FIG. 20 shows in simplified form a fetal heart rate signal with a plurality of baseline regions, acceleration regions and deceleration regions including overhang portions in accordance with a specific example of implementation of the present invention;

FIGS. 21a, 21b and 21c show a representation of a property of the bounded area in accordance with a specific example of implementation of the present invention;

FIG. 22 shows a visual output of the labelled sections of the fetal heart rate in accordance with a specific example of implementation of the present invention;

FIG. 23 shows a computing unit for implementing a heart rate monitoring apparatus in accordance with a specific embodiment of the present invention;

FIG. 24 shows a block diagram of a feature identification unit operative to generate a list of labelled heart rate sections in accordance with an alternative example of implementation of the present invention;

FIG. 25 shows in simplified form a signal diagram of the foetal heart rate signal being processed by the deceleration/acceleration detection 2450 phase of the feature identification unit of FIG. 24;

FIG. 26 shows a frequency response of a filter suitable for use in the deceleration/acceleration detection 2450 phase of the feature identification unit of FIG. 24 in accordance with a specific example of implementation of the present invention;

FIG. 27 is a signal diagram depicting peak detection using quantization in accordance with a specific example of implementation of the present invention;

FIG. 28 is a diagram of a foetal heart rate signal illustrating two categories of decelerations in accordance with a specific example of implementation of the present invention;

FIGS. 29a and 29b are signal diagrams of foetal heart rate signals illustrating various feature measures in accordance with a specific example of implementation of the present invention;

FIG. 30 shows probability distributions for accelerations and decelerations in a foetal heart rate signal.

Other aspects and features of the present invention will become apparent to those ordinarily skilled in the art upon review of the following description of specific embodiments of the invention in conjunction with the accompanying figures.

### DETAILED DESCRIPTION

The detailed description below refers to a fetal heart rate monitoring system. However, the skilled person in the art will appreciate that the processes described herein below may also be applied to heart rate monitoring systems for adult patients without departing from the spirit of the invention.

System Overview

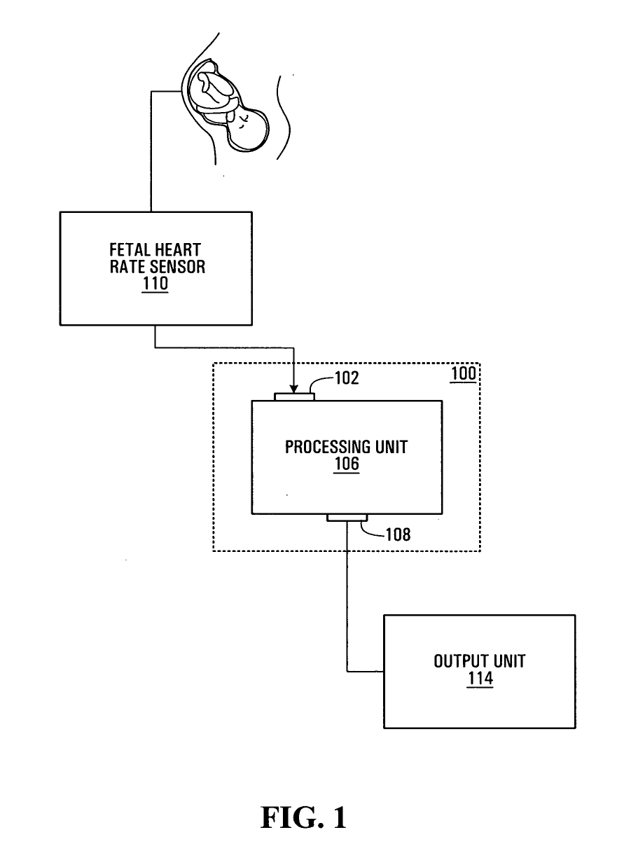

With reference to FIG. 1, there is shown a fetal heart-rate monitoring system in accordance with a specific example of implementation of the present invention. In the embodiment shown, the fetal heart-rate monitoring system comprises a fetal heart-rate sensor 110, an apparatus suitable for monitoring the condition of a fetus 100 and an output unit 114.

The fetal heart-rate sensor 110 is operative to detect the heart rate of a fetus in-utero, which is also referred to as a fetus in the womb. The fetal heart rate sensor 110 samples the fetal heart rate at a certain pre-determined frequency in order to generate a signal indicative of the fetal heart rate. In a specific implementation, the fetal heart rate signal includes a sequence of sample points each being indicative of the number heart beats per minute at a given point in time. Fetal heart rate sensors are well known in the art to which this invention pertains and any suitable sensor for detecting a fetal heart rate may be used without departing from the spirit of the invention. As such, fetal heart rate sensors will not be described further herein.

In a non-limiting example of implementation, the fetal monitoring system further includes a sensor (not shown) for monitoring a mother's uterine activity (TOCO). The sensor samples the mother's contraction pattern at a certain pre-determined frequency to generate the signal indicative of the uterine activity. Sensors for monitoring uterine activity are well known in the art to which this invention pertains and any suitable sensor may be used without detracting from the spirit of the invention. As such, uterine activity sensors will not be described further here.

The apparatus 100 for processing the fetal heart rate signal includes an input 102 for receiving the signal from the fetal heart rate sensor 110, a processing unit 106 for processing the signal in order to generate meaningful data including feature events in the fetal heart rate signal, and an output 108 for releasing the meaningful data to an output unit 114. More specifically, the heart rate feature events include baseline events, acceleration events and deceleration events. The processing unit 106 is also adapted for deriving variability measures associated with the fetal heart rate signal.

The output unit 114 receives the meaningful data indicative of the features of the heart rate signal generated by the processing unit 106 and provides the data to the physician or other health care professional in a visual and/or audio format. The output unit 114 may be in the form of a display screen, a paper print out or any other suitable device for conveying to the physician, or other health care professional, the data indicative of the features of the heart rate signal.

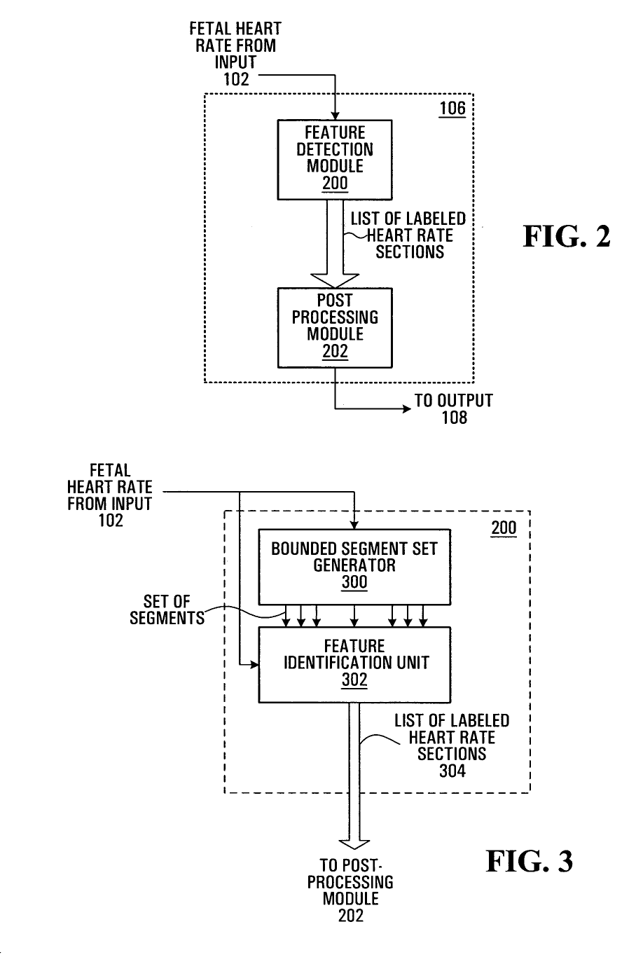

A specific example of a processing unit 106 in accordance with the present invention is shown in more detail in FIG. 2. Processing unit 106 comprises a feature detection module 200 and a post-processing module 202. These two modules are operative to process the fetal heart rate signal received from input 102, determine the feature events of the fetal heart rate signal, process the feature event to generate meaningful information and release the information through output 108.

The feature detection module 200 is operative to process the fetal heart rate signal received from input 102 in order to identify the feature events within the fetal heart rate signal. More specifically, the feature detection module 200 is operative to generate a list of labelled heart rate signal sections where each section is associated with a respective heart rate feature of either a baseline event, an acceleration event or a deceleration event. It will be appreciated that, in practical implementations of the feature detection module, the fetal heart rate signal may include artifactual data samples which are not part of the fetal heart rate per se. Such artifactual data samples may be ignored by the feature detection module 200 without detracting from the feature of the invention.

The post-processing module 202 is coupled to the feature detection module 200 and receives the list of labelled heart rate signal sections from the feature detection module 200. Optionally, certain sections in the list of labelled heart rate signal sections may be labeled as unclassified sections when the sections could not be assigned to a respective heart rate feature event. The post-processing module 202 processes the list of labelled heart rate signal sections in order to generate meaningful data for the user. For example, the post processing module 202 can produce a diagnostic output, a graphical output displaying the heart rate signals with labels identifying selected feature events, or any other suitable useful information that can help the user to better assess the characteristics of the fetal heart rate. It will be readily appreciated that the post processing module 202 can be any device or apparatus that processes the list of labelled heart rate signal sections in order to provide meaningful information to the user.

Feature Detection Module 200

The feature detection module 200 processes the fetal heart rate signal to locate segments of the heart rate signal that are substantially uniform and likely to be associated with a flat region or non-uniform and likely to be representative of an acceleration or deceleration. In a specific implementation, this is achieved by enclosing portions of the fetal heart rate signal within respective bounded areas and processing the signal within the bounded area to identify a possible feature event.

Shown in FIG. 3 is a feature detection module 200 in accordance with a specific example of implementation. Feature detection module 200 includes a bounded segment set generator 300 and a feature identification unit 302.

More specifically, the bounded segment set generator 300 receives the signal indicative of the fetal heart rate signal that includes a sequence of sample points from input 102, and processes the signal in order to generate a set of segments. Each segment in the set of segments is formed by enclosing a portion of the fetal heart rate signal in a respective bounded area. Each bounded area begins at a certain sample point of the heart rate signal and ends at a certain sample point of the heart rate signal. For the purposes of this description, the sample point at which the bounded area begins will be called the start sample point and the sample point at which the bounded area ends will be called the end sample point. All the sample points of the fetal heart rate signal between the start sample point and the end sample point lie within the bounded area. Each bounded area is characterized by a certain length indicative of the duration of the heart rate signal that it contains, as well as a width indicative of the allowable heart rate signal excursion within the bounded area. Optionally, each bounded area may further be characterized by a certain allowable drift in the heart rate signal that it contains. A bounded area can have any suitable shape that permits limiting the allowable excursion and drift in the heart rate signal that the bounded area contains. In a first specific non-limiting example of implementation, the bounded area is in the form of a trapezoid where the two parallel portions define the allowable excursion of the heart rate signal. In a second specific non-limiting example of implementation, the bounded area is in the form of a parallelogram.

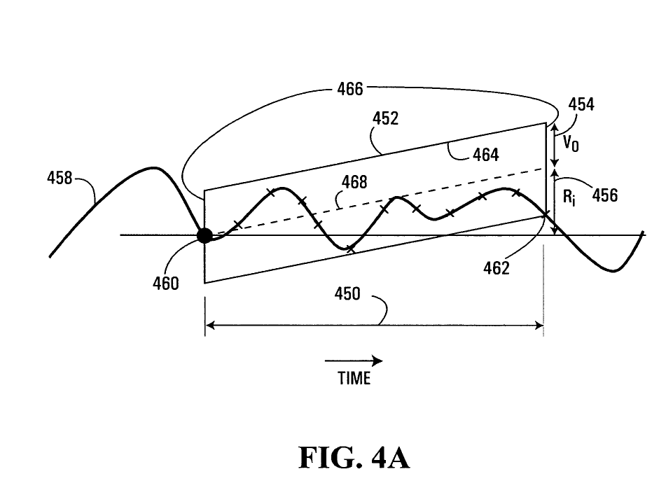

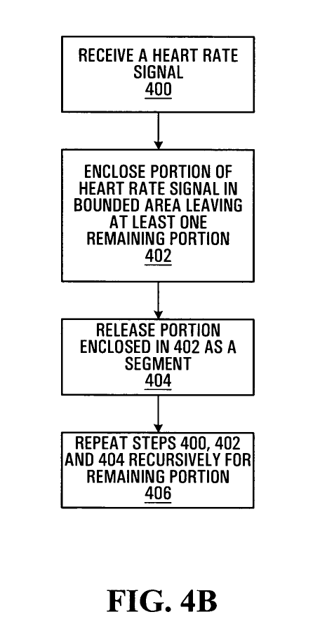

FIG. 4a illustrates in simplified form a portion 450 of a heart rate signal 458 enclosed within a bounded area 452. As shown, the bounded area 452 commences at a start sample point 460 of the heart rate signal and terminates at an end sample point 462 of the heart rate signal. The sample points of the heart rate signal between the start sample point 460 and the end sample point 462 lie within bounded area 452. In the example shown in FIG. 4a, the bounded area 452 is in the form of a parallelogram. The width of the parallelogram is 2*V₀. V₀ may be a fixed value or may be a function of the characteristics of the heart rate signal without detracting from the spirit of the invention. In a non-limiting example, V₀ is conditioned at least in part on the basis of the variability of at least part of the heart rate signal. The sides 464 of the parallelogram along the time axis lie about a line 468 indicative of a polynomial approximation of the sample points of the heart rate signal lying within the bounded area 452. In a non-limiting example, the polynomial approximation of the sample points between the start sample point 460 and end sample point 462 is a line of best fit 468. The line of best fit can be obtained using well-known techniques such as linear regression for example. In a specific non-limiting implementation, the sides 464 of the bounded area 452 are substantively parallel to the line of best fit 468 and are equidistant therefrom. The distance of each of the sides 464 of the bounded area 452 from the line of best-fit 468 is V₀. The line of the best-fit starts at the start sample point 460 and deviates from the start sample point 460 by no more than a certain drift value Rᵢ. Although the sides 464 are shown as being parallel to the line of best-fit and equidistant therefrom, this is not necessary for the purposes of the invention.

The manner in which the length, width (V₀) and drift (Rᵢ) characteristics of a given bounded area may be determined will be described further on in the specification.

Once the bounded segment set generator 300 has generated a list of segments, a signal indicative of a set of segments is released. As a non-limiting example, the signal indicative of the set of segments includes for each segment, a segment identifier, the segment start sample point and the segment end sample point. A more detailed description of the bounded segment set generator 300 is provided further on in the description. The feature identification unit 302 is coupled to the bounded segment set generator 300 and receives the signal indicative of the set of segments generated by the bounded segment set generator 300. In addition, the feature identification unit 302 also receives the signal indicative of the fetal heart rate from input 102. The feature identification unit 302 processes the set of segments and the signal indicative of the fetal heart rate in order to generate a list of labeled heart rate signal sections 304. Each section in the list 304 is associated with a respective heart rate feature event such as an acceleration event, a deceleration event or a baseline event. Optionally, certain sections in the list 304 may be labeled as unclassified sections where the feature identification unit 302 could not classify the sections to a respective heart rate feature event. In a specific implementation, the list of labeled heart rate signal sections includes a plurality of data elements, each data element being associated with a respective section and including a label component indicative of a heart rate feature such as an acceleration event, a deceleration event or a baseline event.

The feature identification unit 302 then releases the list of labeled heart rate sections 304 to the post processing module 202.

I. Bounded Segment Set Generator 300

In a specific example of implementation, the bounded segment set generator 300 makes use of a recursive approach to process the received heart rate signal to generate a set of segments. It will be appreciated that the bounded segment set generator 300 may make use of a suitable non-recursive process to generate a set of segments without detracting from the spirit of the invention. Such a non-recursive process will become readily apparent to the person skilled in the art in light of the description below.

FIG. 4b outlines a specific example of the recursive process implemented by the bounded segment set generator 300. At step 400 the bounded segment set generator receives an interval of the signal indicative of the fetal heart rate from input 102. At step 402 the bounded segment set generator 300 encloses a portion of the interval of fetal heart rate signal within a bounded area leaving a portion of the interval of fetal heart rate signal remaining outside the bounded area. The remaining portion includes sample points of the heart rate signal excluded from the enclosed portion. The portion of the fetal heart rate signal that is enclosed within the bounded area is the longest portion of the interval of fetal heart rate signal that will fit within a bounded area defined by certain criteria of drift and excursion. The determination of the bounded area will be explained in more detail further on in the specification.

At step 404 the bounded segment set generator 300 releases as a segment the portion of the heart rate signal that was enclosed in the bounded area. After performing these steps, at step 406, the bounded segment set generator 300 receives the remaining portion(s) of the fetal heart rate signal at step 400, and repeats steps 402 and 404 on those portion(s) of the fetal heart rate signal remaining outside the bounded area. As such, the second segment is the longest portion of the fetal heart rate signal found in the remaining portion of the interval of the fetal heart rate signal where the second segment is enclosed within a second bounded area. Steps 400, 402 and 404 are repeated recursively on the remaining portions of the heart rate signal until there is no remaining portion of the heart rate signal left to process, or until a pre-defined criteria is met. In a specific example of implementation, the pre-defined criteria is met when the remaining portion(s) of the heart rate signal has a number of sample points below a pre-determined threshold number of sample points, or if no box can be found that is longer than a minimum threshold.

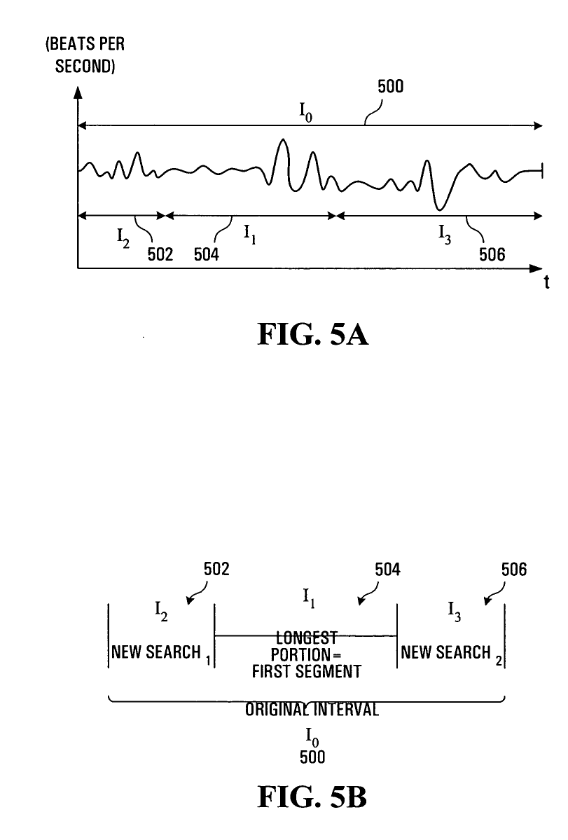

FIGS. 5A and 5B provide simplified examples of a visual representation of the process performed by the bounded segment set generator 300. Shown in FIG. 5A is an interval I₀ 500 of the fetal heart rate signal that is received by the bounded segment set generator 300 at step 400. It should be understood that interval I₀ 500 can be the complete fetal heart rate signal, or a part of the fetal heart rate signal. At step 402, the bounded segment set generator 300 encloses the largest portion of the interval I₀ 500 of fetal heart rate signal that will fit within a bounded area defined by certain criteria of drift and excursion. In the case shown in FIG. 5A, interval I₁ 504 is the largest portion of the interval I₀ 500 of fetal heart rate signal that will fit within a bounded area, and intervals I₂ 502 and I₃ 506 are the remaining portions of the interval of fetal heart rate signal. As such, at step 404, interval I₁ 504 will be released as a segment and the bounded segment set generator 300 will repeat steps 400, 402 and 404 on intervals I₂ 502 and I₃ 506 recursively in order to find a second segment, a third segment and so on.

Shown in FIG. 5B, interval I₁ 504 is the longest portion found in I₀ 500, and as such is the first segment. A new search will be performed in intervals I₂ 502 and I₃ 506 in order to find the second longest portion of interval I₀ 500 that can be enclosed in a bounded area.

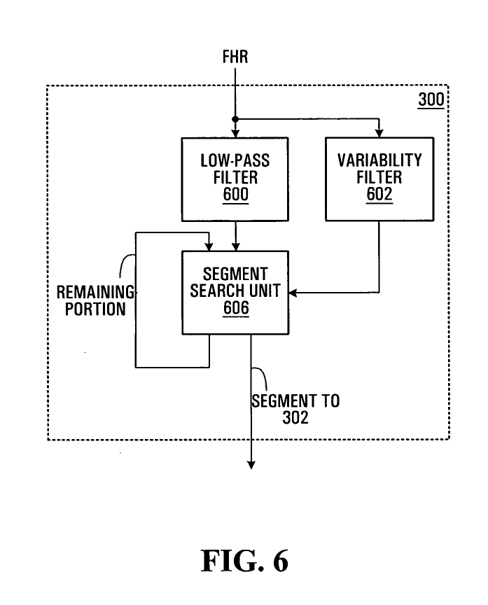

A more detailed description of a specific example of implementation of the bounded segment set generator 300 will be described below with reference to FIGS. 6 through 14B.

A functional diagram of the bounded segment set generator 300 in accordance with a specific example of the present invention is shown in FIG. 6. In the specific example of implementation shown, bounded segment set generator 300 includes a low-pass filter 600, a variability filter 602 and a segment search unit 606.

The low-pass filter 600 receives the fetal heart rate signal and generates a filtered version of the heart rate signal. The filtered version of the heart rate signal can be used to locate segments likely to be associated with a common feature event since the features to be detected within the fetal heart rate signal, namely baseline events, acceleration events and deceleration events, are generally of low frequency relative to the sampling rate of the heart rate signal. The resulting filtered heart rate signal is smoother that the original signal since high-frequency components of the signal have been removed, and thus the filtered signal is more amenable to subsequent feature detection. It is to be appreciated that the low-pass filter 600 may be omitted from the bounded segment generator 300 without detracting from the spirit of the invention.

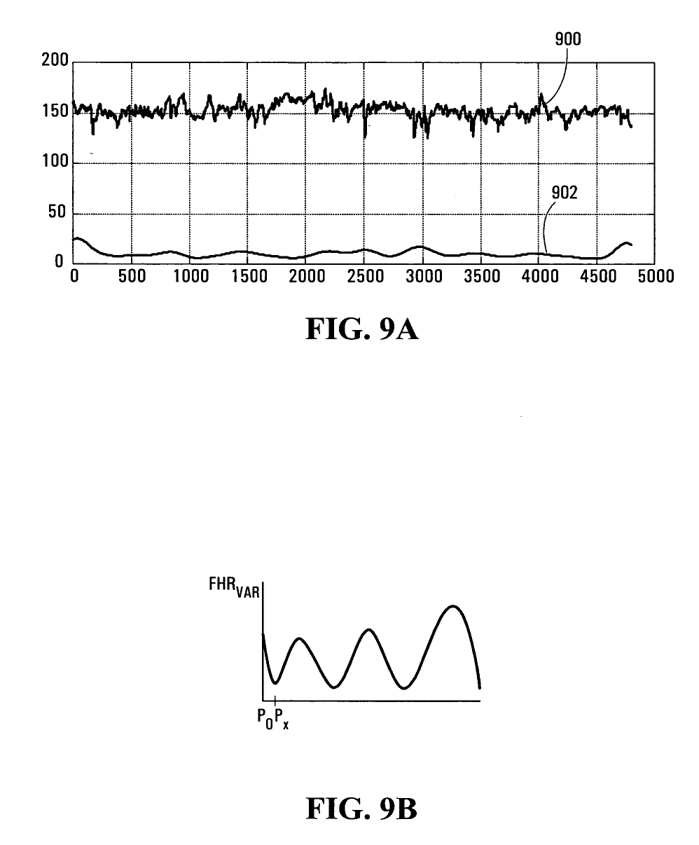

The variability filter 602 receives the fetal heart rate signal and generates a variability signal indicative of the variability of the heart rate signal over time. Any suitable method well known to the person skilled in the art may be used to derive a variability signal. For example, the variability signal can be obtained by applying a high pass filter to the fetal heart rate signal then full-wave rectifying the signal before low-pass filtering the result. FIG. 9b illustrates in graphical format a variability signal associated with a fetal heart rate signal. FIG. 9a illustrates in graphical format a portion of a fetal heart rate signal 900 and associated variability measure 902.

Once the fetal heart rate signal has been processed by the low pass filter 600 and by the variability filter 602, the resulting filtered heart rate signal and variability signal are provided to the segment search unit 606. The segment search unit 606 processes the filtered heart rate signal and variability signal to generate a set of segments. More specifically, the segment search unit 606 is looking for portions of the fetal heart rate signal that are characterised by a relatively constant fetal heart rate level, while allowing for local variability and long term drift. In a specific example, the search includes locating the longest portions of the fetal heart rate signal that can be enclosed within a bounded area of maximum length, limited height and limited slope. For the purpose of this specification, this bounded area will be referred to as the “longest bounded area”.

It will be appreciated that a bounded area can commence at any sample point P₀ of the fetal heart rate signal. Therefore, in a specific example of implementation, the longest bounded area for a given interval of fetal heart rate can be determined by firstly calculating a bounded area for each sample point on the given interval of fetal heart rate signal, and then comparing all the bounded areas to determine which bounded area along the given interval is the longest. The portion of fetal heart rate signal within this longest bounded area is then selected as a segment. The search then continues recursively on the portion (or portions) of the fetal heart rate signal remaining outside the longest bounded area to locate a second longest bounded area, a third longest bounded area and so on. The set of segments is released for further processing by the feature identification unit 302.

In a specific non-limiting example, the bounded areas at the sample points are determined using a process, which for the purposes of this specification will be called the “longest bounded area algorithm”.

The Longest Bounded Area Algorithm

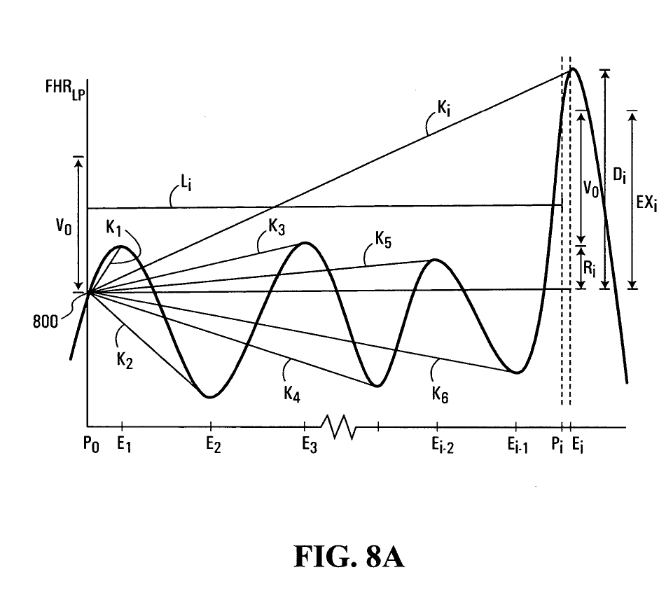

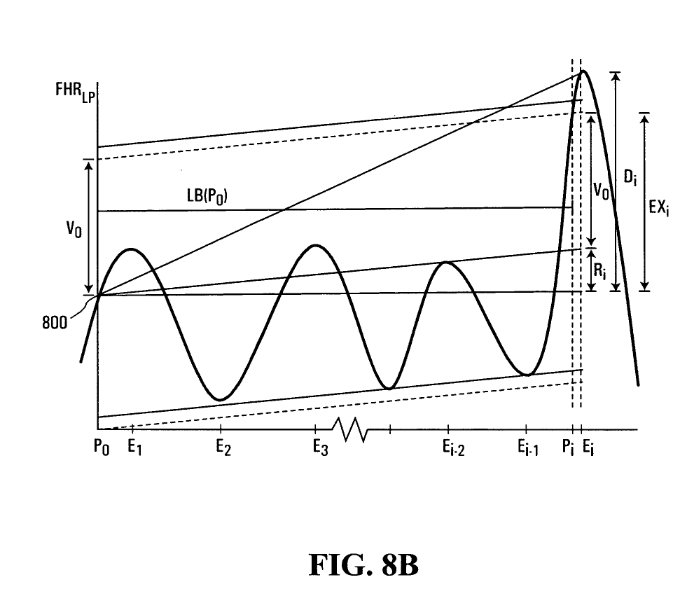

The longest bounded area algorithm will now be described with respect to FIGS. 8a and 8b. Firstly, as shown in FIG. 8a, a line is drawn from a start sample point P₀ 800 to each subsequent extrema point Eⱼ (for j=1, 2, 3, . . . , i−1, i) on the given interval of the fetal heart rate signal being processed. For each line, labelled K₁; K₂; K₃; K₄; K₅; K₆; . . . ; Kᵢ, the height difference Dⱼ and the length Lⱼ between the start sample point P₀ and the extrema point Eⱼ are calculated. Mathematically, these calculations can be expressed as follows:

Dⱼ=FHR(P₀)−FHR(Eⱼ)

Lⱼ=P₀−Eⱼ Equation 1

for j=1, 2, 3, . . . , i−1, i

Once this has been done, the maximal allowable excursion EXⱼ is calculated for each extrema Eⱼ where j=1, 2, 3, . . . , i−1, i. The maximum allowable excursion EXⱼ is a function of V₀ and the maximum allowable drift value Rⱼ at sample point Eⱼ. FIG. 8a illustrates V₀ and the maximum allowable drift Rᵢ at point Eᵢ. As indicated previously, V₀ is maximum allowable distance from a line of best-fit of the samples of the fetal rate signal. In a non-limiting implementation V₀ is a function of the variability of the fetal rate signal.

In a non-limiting implementation, V₀ is a piecewise linear function of Pₓ, which is the closest local minimum of the fetal heart rate signal variability in the neighbourhood of point P₀, as shown in FIG. 9b. The heart rate signal variability was derived by variability filter 602 described in connection with FIG. 6 of the drawings. Mathematically, V₀ can be expressed as follows:

V₀=K_{V}{min[FHR_{VAR}(Pₓ)]} Equation 2

Where K_{V} is a piece-wise linear function and Pₓ is in the neighbourhood of point P₀.

The maximum allowable drift Rⱼ for each segment P₀Eⱼ is a piece-wise linear function of the length Lⱼ. Mathematically, the maximum allowable drift Rⱼ at a given extrema Eⱼ can be expressed as follows:

Rⱼ=Kᵣ{Lⱼ} Equation 3

for j=1, 2, 3, . . . , i−1, i

Where Kᵣ is a piece-wise linear function.

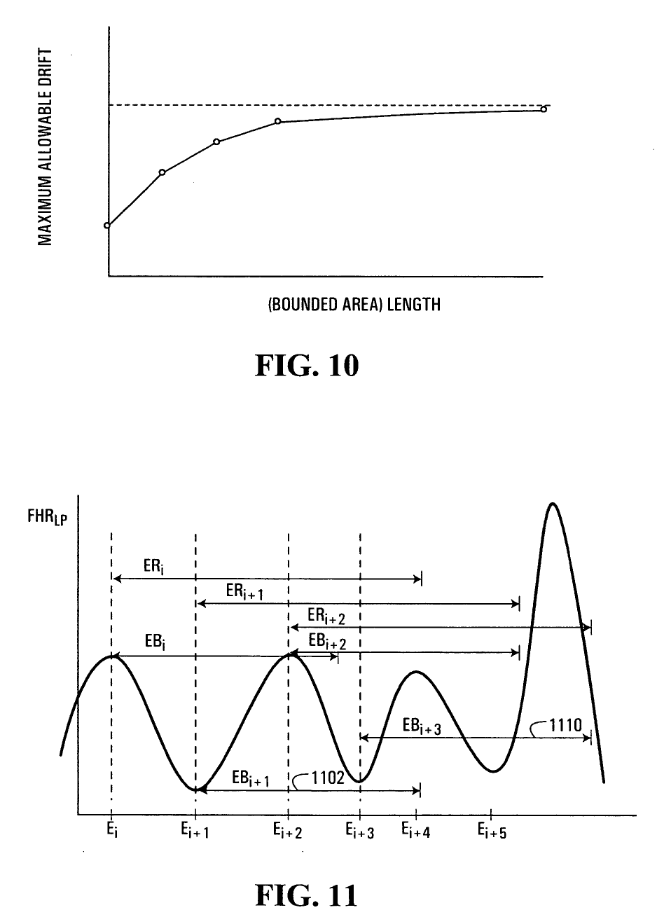

FIG. 10 shows an example of the function Kᵣ{Lⱼ} where the function for drift asymptotically approaches a maximum drift Rₘₐₓ as the length Lⱼ between P₀ and Eⱼ increases.

The maximal excursion EXⱼ at extrema Eⱼ is the sum of V₀ and Drift. Mathematically, the maximum maximal excursion EXⱼ can be expressed as follows:

EXⱼ=V₀+Rⱼ Equation 4

for j=1, 2, 3, . . . , i−1, i

As such, once the maximal excursion EXⱼ at extrema Eⱼ is calculated, it is compared with the Dⱼ for the associated extrema point Eᵢ, in an order starting with the closest extrema point to P₀. When the first extrema point Eⱼ is found whose Dᵢ exceeds the maximum excursion EXⱼ, an original corridor is constructed from P₀ to the position where the maximum excursion crosses the fetal heart rate signal. As shown in FIG. 8b, extrema point Eᵢ is the first extrema for which Dᵢ>EXᵢ. The width of the corridor is 2*V₀ and the drift is Rᵢ. All extrema points preceding Eᵢ should be enclosed within the original corridor. The original corridor is shown in dotted lines in FIG. 8b. The original corridor is then shifted such as to try to enclose even more of the fetal heart rate signal within the corridor. However, the bounded area can only be shifted from the original corridor if all extrema points preceding Eᵢ are enclosed within the shifted corridor. The shifted corridor is shown in solid lines in FIG. 8b.

If extrema Eᵢ falls outside the shifted corridor, then that corridor is the longest bounded area for starting point P₀. In the example shown in FIG. 8b, Eᵢ falls outside the corridor and the last sample point within the corridor is Pᵢ. Therefore, the longest bounded area starting at sample point P₀ ends at sample point Pᵢ. However, if extrema Eᵢ fits within the shifted corridor, then the search continues with a subsequent extrema. In the specific example of implementation shown in FIG. 8b, the bounded area is a parallelogram with the upper and lower boundaries of the corridor constituting two parallel sides of the parallelogram and the vertical lines at P₀ and Pᵢ constituting the remaining two parallel sides of the parallelogram. It should however, be understood that the bounded area may be of any suitable shape, such as a rectangle or trapezoid, for example. A rectangle would be the case where the maximum allowable drift Rⱼ=0.

Once the longest portion enclosed within a bounded area has been located for each sample point, the length of the longest portions are compared and the longest portion is selected as a segment to be released by the segment search unit 606 (shown in FIG. 6). The selection of the longest portions is then recursively performed on the remaining portions of the heart rate signal.

Refinements to the Longest Bounded Area Algorithm

It should be understood that calculating a longest bounded area from a sample point, as described above, for each sample point in the fetal heart rate signal would be computationally prohibitive due to the number of sample points to be processed. As such, in a specific example of implementation, the longest bounded area is determined using a series of steps that reduces the amount of computations required. The first step in finding the longest bounded area for a given interval of heart rate signal is to calculate a bounded area of maximum length only for sample points that corresponds to local extrema sample points in the given interval of heart rate signal. Following this, sample points between two extremas can be selectively processed using properties of the bounded areas to locate the longest bounded area. This process will generally reduce the number of bounded areas calculated. For the purposes of this specification, each of the bounded areas starting from a local extrema point will be called an “extrema bounded area”.

It will become apparent to the person skilled in the art in light of the present description that a property of the bounded areas is that a second longest bounded area will extend at least as far as a first longest bounded area if the first longest bounded area began before the second bounded area, assuming the excursion criteria (i.e. variability and drift) remain constant. For the purpose of this description, this property will be referred to as “the longest bounded area rule”. A specific example of this property is illustrated in FIG. 21A. None of the bounded areas beginning at a subsequent point end before the end of the previous bounded area. FIG. 21B shows two bounded areas where Box 2 ends before then end of Box 1 indicating that Box 2 is not the longest box including the starting point of Box 2.

Referring to FIG. 21C, this property can be illustrated by considering that the bounded area LBA(P₀) commencing at a point P₀, is defined as a bounded area of maximal length L₀ and restricted excursion V₀+Rᵢ that contains all the fetal heart rate samples over the bounded area. It remains true that the longest bounded area over the interval (P₀+Δp) to Pe, where Pe=P₀+L₀, extends as far as Pe if the same excursion criteria is applied to the subset of the points in the interval (P₀+Δp) to Pe. Application of the same excursion criteria is valid for a sufficiently small neighbourhood Δp. This gives a lower bound, therefore, on the length of the LBA(P0+Δp). It follows that no longest bounded area can end before the end of a preceding longest bounded area provided the excursion criteria (i.e. variability and drift) remain constant.

The above described property of the bounded areas, herein referred to as the longest bounded area rule, can be used in practical implementations of the longest bounded area algorithm to reduce the search space and locate the longest bounded areas for portions of the fetal heart rate signal using fewer mathematical computations.

Many other refinements may be made to the longest bounded area algorithm to improve the efficiency of the search and reduce the mathematical complexity of the search without detracting from the spirit of the invention.

Segment Search Unit 606

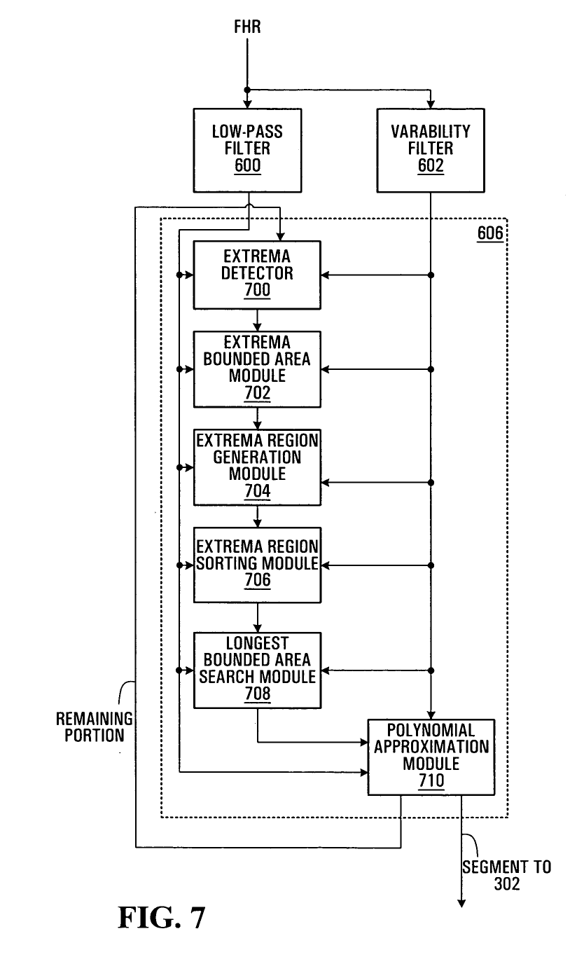

FIG. 7 shows a segment search unit 606 in accordance with a specific example of implementation of the present invention. In the specific example, the segment search unit 606 includes an extrema detector module 700, an extrema bounded area module 702, an extrema region generation module 704, an extrema region sorting module 706, a longest bounded area search module 708 and a polynomial approximation module 710. The function of each of these modules will be described in more detail below.

a) Extrema Detector Module 700

In the specific example of implementation shown, the extrema detector module 700 receives the fetal heart rate signal from the low pass filter 600. The extrema detector module 700 of the segment search unit 606 locates the local extrema points Eⱼ in the interval of fetal heart rate signal. The local extrema points can be detected using any suitable technique known in the art.

Once the local extrema points have been determined, the extrema detector module 700 releases a list indicative of the local extrema points.

b) Extrema Bounded Area Module 702

In the specific example of implementation shown, the extrema bounded area module 702 is coupled to the extrema detector module 700 and is adapted to receive the signal indicative of the list of local extrema points for an interval of the fetal heart rate signal. In addition, the extrema bounded area module 702 is adapted to receive the filtered fetal heart rate signal from the low pass filter 600 and the variability signal from variability filter 602. Once these signals have been received, the extrema bounded area module 702 calculates an extrema bounded area starting at each local extrema point Eⱼ. The extrema bounded areas are found using the longest bounded area algorithm described above, with the start sample points being the local extrema points in the list of local extrema points.

Once the set of extrema bounded areas have been found for the given interval of fetal heart rate signal, the extrema bounded area module outputs a signal indicative of the set of extrema bounded areas.

c) Extrema Region Generation Module 704

In the specific example of implementation shown, the extrema region generation module 704 is coupled to both the extrema detector module 700 and the extrema bounded area module 702. As such, the extrema region generation module 704 is adapted to receive the signal indicative of the list of local extrema points as well as the signal indicative of the set extrema bounded areas. In addition, the extrema region generation module 704 is adapted to receive the filtered fetal heart rate signal from the low pass filter 600 and the variability signal from variability filter 602.

The extrema region generation module 704 of segment search unit 606 processes the extrema bounded area to generate a set of extrema regions. For the purpose of this specification, an extrema region is the interval extending from a first extrema point to the end sample point of the extrema bounded area that starts at the extrema point directly subsequent to the first extrema point.

FIG. 11 shows a simplified specific example of a graphical representation of the extrema regions for an interval of the fetal heart rate signal. Shown in the specific example are four extrema bounded areas which are labelled EBᵢ, EBᵢ₊₁, EBᵢ₊₂, EBᵢ₊₃, respectively. In addition, their associated extrema regions are also shown and are labelled ERᵢ, ERᵢ₊₁ and ERᵢ₊₂. Extrema region ERᵢ starts at the extrema point Eᵢ, which is also the commencement point of extrema bounded area EBᵢ, and extends to the end of extrema bounded area EBᵢ₊₁. Extrema region ERᵢ₊₁ starts at the extrema point Eᵢ₊₁, which is also the commencement point of extrema bounded area EBᵢ₊₁, and extends to the end of extrema bounded area EBᵢ₊₂. Finally, extrema region ERᵢ₊₂ starts at the extrema point Eᵢ₊₂, which is also the commencement point of extrema bounded area EBᵢ₊₂, and extends to the end of extrema bounded area EBᵢ₊₃.

Once the set of extrema regions has been determined for the given interval of fetal heart rate signal, the extrema region generation module 704 outputs a signal indicative of a set of extrema regions ERⱼ.

d) Extrema Region Sorting Module 706

In the specific example of implementation shown, the extrema region sorting module 706 receives the signal indicative of the set of extrema regions from the extrema region generation module 704. The extrema region sorting module 706 is operative to sort the extrema regions in order of length. This may be done using any suitable well-known sorting algorithm known to those of skill in the art.

Once the set of extrema regions has been sorted, the extrema region sorting module 706 outputs a signal indicative of the sorted list of extrema regions.

e) Longest Bounded Area Search Module 708

In the specific example of implementation shown, the longest bounded area search module 708 receives the signal indicative of the list of sorted extrema regions from the extrema region sorting module 706. In addition, the longest bounded area search module 708 receives the filtered fetal heart rate signal from the low pass filter 600 and the variability filter 602.

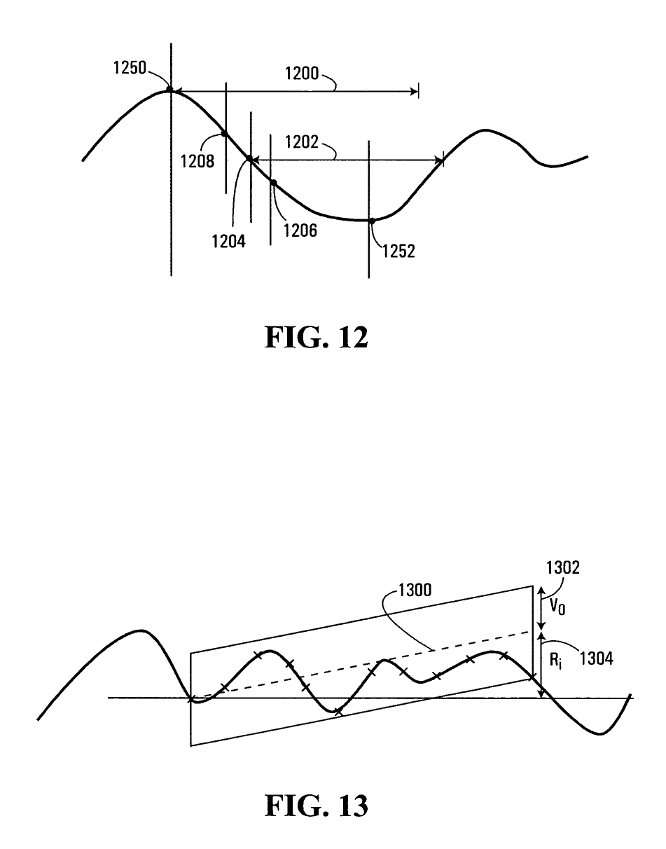

Starting with the longest extrema region in the sorted list of extrema regions and proceeding with the other extrema regions in decreasing order of length, the longest bounded area search module 708 performs a search using the longest bounded area algorithm between the first extrema point and the subsequent extrema point, of each extrema region. The purpose of the search is to find the sample point between the two extrema points that has the longest associated bounded area. The longest bounded area rule may be used to further reduce the search space. In a non-limiting specific implementation, the search is effected by recursively bisecting the interval between the two extrema points and finding an associated bounded area at the midpoint until the sample point having the maximum bounded area length is found. This is best described with reference to FIG. 12. As depicted, the extrema region for extrema point 1250 is 1200. The interval between extrema point 1250 and 1252 is then bisected and the longest bounded area at the midpoint 1206 is then determined. At each stage, the interval having the longer bounded area is bisected until there is no more interval to bisect (i.e. on the order of log₂n trial starting points are tested between two extrema points n samples apart).

Once the longest bounded area for the longest extrema region has been found, it becomes the candidate for the longest bounded area for the given interval of the fetal heart rate signal. The longest bounded area is then found for the second longest extrema region in the sorted list of extrema regions. If the longest bounded area for the second longest extrema region is longer than the current candidate for the longest bounded area, then the longest bounded area for the second longest extrema region becomes the candidate for longest bounded area for the given interval of the fetal heart rate signal. However, if the longest bounded area for the second longest extrema region is not longer than the longest bounded area for the longest extrema region, then it is discarded and the longest bounded area candidate remains the same. This process is continued with the other extrema regions until the longest bounded area candidate is longer than the next extrema region to be searched.

Once the longest bounded area search module has determined the longest bounded area candidate, it outputs a signal indicative of the longest bounded area candidate.

f) Polynomial Approximation Module 710

In accordance with a variant, the segment search unit 606 further includes a polynomial approximation module 710. It will be appreciated that the polynomial approximation module 710 may be omitted from the segment search unit 606 without detracting from the spirit of the invention. In the specific example of implementation shown in FIG. 7 the polynomial approximation module 710 receives from the longest bounded area search module 708 the signal indicative of the longest bounded area candidate. In addition, the polynomial approximation module 710 also receives the filtered fetal heart rate signal from the low pass filter 600 and the variability signal from variability filter 602.

The polynomial approximation module 710 refines the longest bounded area candidate using polynomial line-fitting. The general purpose is to enclose a portion of the fetal heart rate signal within a bounded area that is subject to local variability and drift restrictions. However, as opposed to the “longest bounded area” algorithm described above, wherein it is the line joining the start sample point Po and an end sample point Pi that must satisfy the variability and drift conditions, for the polynomial line fitting module 710, it is a line of best fit for the longest bounded area candidate that must satisfy the variability and drift criteria. In other words, the height difference Dᵢ between the start and end sample points of the line of best fit, must not exceed the maximal excursion EXᵢ defined by the previously determined variability and drift criterion. It should be noted that the V₀ and drift Rᵢ limits need not be recalculated and that the limits associated with the “longest bounded area” algorithm for that start point may be used to reduce computational requirements.

FIG. 13 shows in simplified form a signal diagram of a heart rate signal having a portion enclosed within a longest bounded heart rate region and a polynomial-fitted line shown in dotted lines in accordance with a specific example of implementation of the present invention.

For the purposes of this description, the following test be referred to as the “polynomial bounded area criterion”:

“Polynomial Bounded Area Criterion”

If Dᵢ>EXᵢ

then test failed

otherwise test passed;

where:

Dᵢ=FHR(P₀)−FHR(Pᵢ)

EXᵢ=V₀+Rᵢ

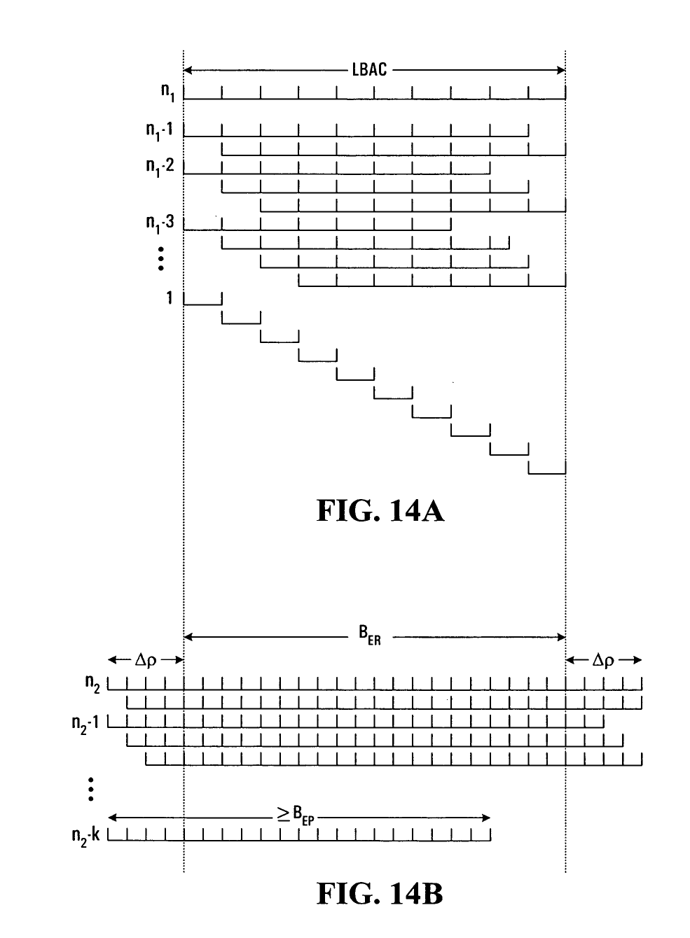

In a non-limiting implementation, a two-step line fitting procedure is used to determine a longest polynomial bounded area, wherein with each step the resolution increases. The first step of this method will be described with reference to FIG. 14A, wherein the longest bounded area candidate is labelled LBAC. Longest bounded area candidate LBAC is divided into a fixed number of sub-intervals. The sub-intervals can be of equal length or un-equal length. The calculation of the fitted line is restricted to regions that are enclosed by points on the subinterval boundaries. For example, the line of best fit is first determined for the whole region LBAC and is then analysed to see if it satisfies the “polynomial bounded area” criterion. If not, then the line of best fit is determined for successively smaller regions. As can be seen in FIG. 14A, at pass n1 the line of best fit for the whole region LBAC is calculated. Then, if that line of best fit does not satisfy the “polynomial bounded area” criterion, at pass n1−1 the line of best fit is calculated for the region of LBAC minus one subinterval on the left, and then for the region LBAC minus one subinterval on the right. Then at pass n1−2 the line of best fit is calculated for the region of LBAC minus two subintervals on the left, and then for the region LBAC minus two subintervals on the right. This process continues until a line of best fit satisfies the “polynomial bounded area criterion” and the longest polynomial bounded area is found.

The purpose of step two, which will now be described with reference to FIG. 14B, is to find a polynomial bounded area that is longer than the longest polynomial bounded area found in step one. As can be seen, the longest bounded area candidate LBAC is divided into a plurality of subintervals of a finer resolution than those of step one and is widened by an amount Δp on either side. As such, the search space is widened. At this stage, the subintervals are searched in the same way as in step one, starting with the region LBAC+2Δp, with the goal of finding a polynomial bounded area larger than that found in step one. However, the search ends when the polynomial bounded area found in step two equals that of the polynomial bounded area found in step one. The polynomial bounded area found in step two then becomes the longest bounded area for the interval of fetal heart rate signal that was being searched.

As such, the portion of fetal heart rate signal contained within the longest bounded area is the first segment.

At this stage, the polynomial approximation unit 710 releases the first segment to feature identification unit 302, and outputs the remaining portions of the fetal heart rate signal back to the segment search unit 606.

This process is then repeated recursively for the portions of the fetal heart rate signal that are not released as a segment. As such, the recursive process generates a set of segments that are output to the feature identification unit 302.

II. Feature Identification Unit 302

As mentioned previously, feature identification unit 302 receives from the bounded segment set generator 300 a set of segments generated by bounded segment set generator 300. Each segment that is transmitted from the bounded segment set generator 300 to feature identification unit 302 represents a candidate baseline feature event. The feature identification unit 302 processes each segment in the set of segments and generates a plurality of sections, each section being indicative of a heart rate feature selected from the set consisting of an acceleration event, a deceleration event and a baseline event. Each section is associated with a respective label data element indicative of a corresponding feature event such as a baseline, an acceleration or a deceleration. The feature identification unit 302 releases a list of labelled heart rate sections.

### FIRST SPECIFIC EXAMPLE OF IMPLEMENTATION

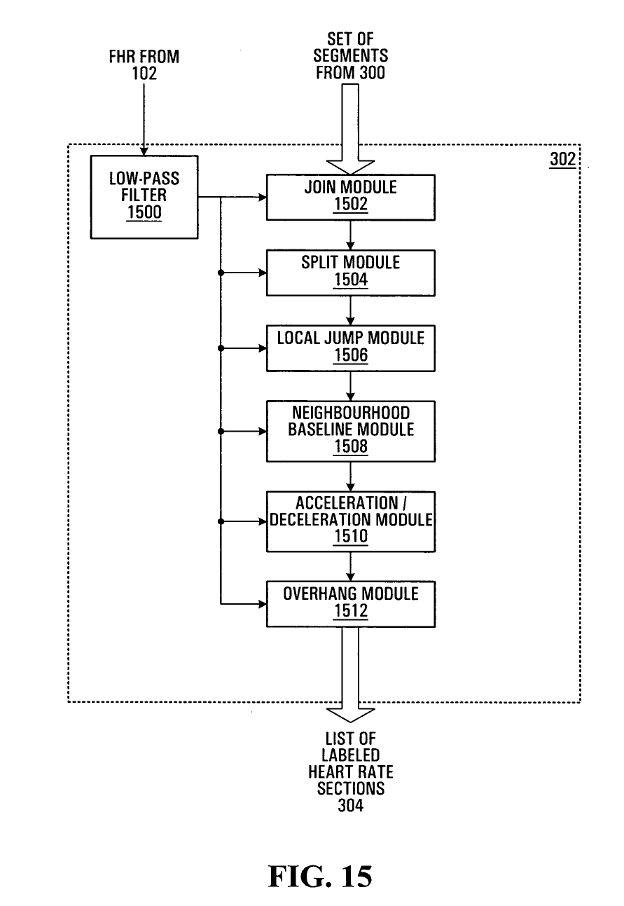

A feature identification unit 302 in accordance with a first specific example of implementation of the present invention is shown in FIG. 15. As shown, feature identification unit 302 receives the set of segments from bounded segment set generator 300 and the fetal heart rate from input 102. The feature identification unit 302 includes a low pass filter 1500, a join module 1502, a split module 1504, a local jump module 1506, a neighborhood baseline module 1508, an acceleration/deceleration module 1510 and an overhang module 1512. Each of these modules is described in greater detail below.

a) Low-Pass Filter 1500

The low-pass filter 1500 processes the fetal heart rate signal to generate a filtered version of the fetal heart rate. The filtered version of the fetal heart rate is then provided to the remaining modules within feature identification unit 302. It will be apparent that the low-pass filter 1500 may also be embodied in the same physical filter as low-pass filter 600 (shown in FIG. 6) without detracting from the spirit of the invention.

b) Join Module 1502

Join module 1502 is adapted to receive the set of segments from bounded segment set generator 300 and the filtered fetal heart rate signal from low-pass filter 1500.

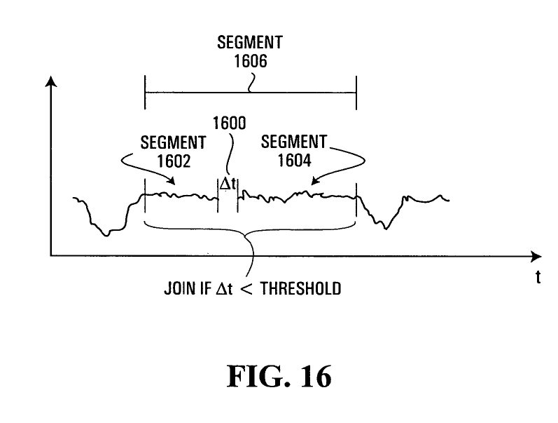

Join module 1502 combines or join two segments of the heart rate signal that are separated by only a short interval. For example, if two neighboring segments are separated by an interval having a duration below a certain proximity threshold duration, the join module combines these two segments to represent them as one longer segment. This is best described with reference to the specific example shown in FIG. 16. As depicted, a first segment 1602 is separated from a second segment 1604 by an interval Δt. If Δt is less than the proximity threshold, then segment 1602 and segment 1604 are combined to form longer segment 1606. In a first non-limiting implementation, the proximity threshold is a constant value. In a second alternative non-limiting implementation, the proximity threshold is a linear function of the local variability.

Multiple passes of the interval of fetal heart rate signal containing the set of segments can be performed. For example, two segments which were joined in the first pass may be joined to a third segment in the second pass. In a specific example of implementation, only two passes of the interval of fetal heart rate signal are performed in order to limit the computational requirements.

The join module then outputs a signal indicative of the new set of segments.

c) Split Module 1504

In the specific example of implementation shown, split module 1504 receives from the join module 1502 the signal indicative of the new set of segments and the filtered fetal heart rate signal from low-pass filter 1500.

The split module 1504 is operative to generate a new line of best fit for all the new segments that were previously two or more separate segments. The split module 1504 reprocesses the new segments by forming a new line of best fit and re-applying the polynomial bounded area algorithm. This is done to ensure that the new line of best fit still fulfils the “Polynomial Bounded Area Criterion”. If the new line-of-best-fit for the new segments does not meet the “Polynomial Bounded Area Criterion”, the newly joined segments are split up.

It will be appreciated that the functionality of the join module 1502 and of the split module 1504 may be combined into a common module without detracting from the spirit of the invention.

As such, the split module 1504 outputs a signal indicative of an updated set of segments.

d) Local Jump Module 1506

In the specific example of implementation shown, the local jump module 1506 receives from the split module 1504 the signal indicative of an updated set of segments and the filtered fetal heart rate signal from low-pass filter 1500.

Local jump module 1506 is adapted to assign to each segment of the updated set of segments an identifier indicative of either one of a baseline event or a non-baseline event.

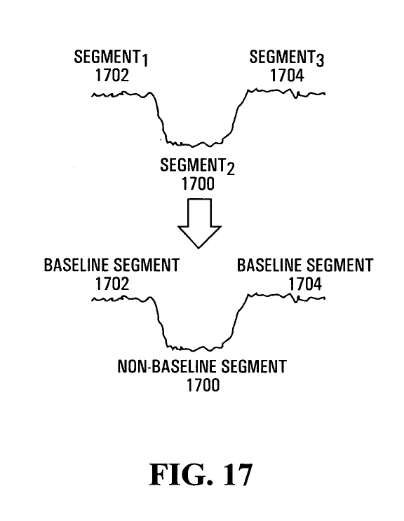

In some areas of the fetal heart rate signal, the sequence of segments may include sharp brief transitions where a segment is briefly displaced by a sharp brief transition, and it is the successive segment that returns to the original level. For example, a given segment may have a line of best fit that is located far from the line of best fit of the previous segment or from the subsequent segment. An example of this is illustrated in FIG. 17. As can be seen, segment 2 1700 might be better classified as a non-baseline event. As such, local jump module 1506 assigns to segment 2 1700 an identifier indicative of a non-baseline event and assigns to segment 1 1702 and segment 1704 respective identifiers indicative of baseline events.

The criteria for establishing that a series of segments includes a sharp brief transition segment are the proximity Δnₘₐₓ and the length Lₘₐₓ of the displaced segment. In a preferred embodiment these thresholds are determined by clinical definitions of the acceleration and deceleration modified by ambiant variability. Therefore, if the proximity and length of the transition segment is greater than the threshold Δnₘₐₓ and the Lₘₐₓ, then that segment is considered to be a non-baseline segment, and it is removed from the list of segments.

The local jump module 1506 then outputs a signal indicative of a list of segments identified as baseline and non-baseline segments.

e) Neighbourhood Baseline Module 1508

In the specific example of implementation shown, the neighbourhood baseline module 1508 receives from the local jump module 1504 the signal indicative of the list of segments identified as baseline and non-baseline segments and the filtered fetal heart rate signal from low-pass filter 1500.

The neighbourhood baseline module 1508 is adapted to further process the list of segments. The purpose of the neighbourhood baseline module 1508 is to extract potential remaining non-baseline segments from the segments classified as baseline.

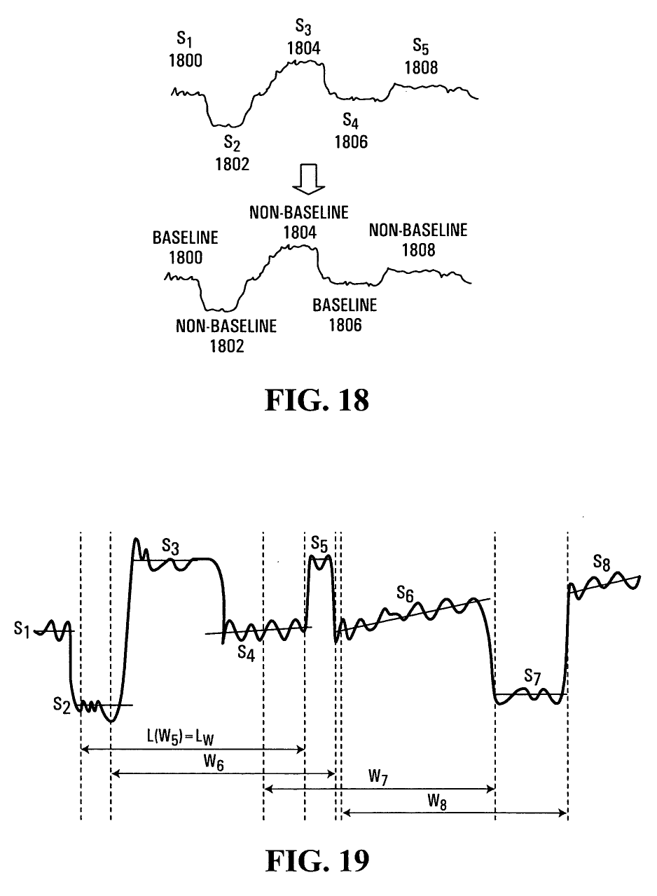

For example, a series of segments can have a plurality of local jumps that occur frequently and that are not sharp enough to be removed by the local jump module 1506, but that may be non-baseline segments nonetheless. This can best be seen with reference to FIG. 18. As such, it can be difficult to determine the true baseline of an interval of fetal heart rate signal. In a specific example of implementation, the neighbourhood baseline module 1508 classifies the level that the majority of segments occur, as being the baseline level. As such the portion of the fetal heart rate signal that is within each baseline segment that occurs at this level can be classified as a section that is indicative of a baseline event.

The process used to find this baseline level can best be described with reference to FIG. 19. Firstly, the list of baseline segments is searched to find a first segment that is proceeded by a window of at least length L_{w}. L_{w} is of a predefined length. If the length of the segment preceding this window is of a length that is greater than a certain threshold Lt, which is also of a predefined length, then that segment is immediately identified as a baseline segment. In the case of FIG. 19, segment S1 is the first segment that is proceeded by a window of a length Lw. However, S1 is not of a length that is greater that a threshold Lt, and therefore is not immediately identified as a baseline segment. As such, the neighbourhood baseline module 1508 proceeds to step two.

At step two, the neighbourhood baseline module 1508 takes the segment proceeding the window and all the segments within the window, and categorises these segments into groups based on matching criteria. The segments that have matching criteria will tend to fall within a corridor that accounts for variability and drift. As such, in FIG. 19, the segments fall into three separate categories:

category a=S2

category b=S3 and S5

category c=S4

The category having the greatest sum of its segments' lengths is considered to be the winning category and the segments within that category are considered to be baseline segments. In the case of FIG. 19, L(S4)>L(S3)+L(S5)>L(2), and therefore, category c is the winning category. As such, segment 4 is considered a baseline segment and the portion of fetal heart rate signal within segment S4 can be classified as a section indicative of a baseline event. Segments that precede the window that do not match the winning category are discarded from the list of baseline segments. (assuming that its length is less than Lt). However, in the case of FIG. 19, S1 has matching criteria with S4 and as such is included in the list of baseline segments. Each subsequent segment S6, S7, S8 etc is compared with the winning group until all the segments have been considered. When processing the final segment, any segment not belonging to the winning group is immediately removed from the baseline segment list. Therefore, segments S1, S4, S6 and S8 would be considered baseline segments. It should be noticed that S6 and S8 have the same matching criteria since a corridor considers drift as well as variability.

The neighbourhood baseline module 1508 outputs a signal indicative of the final set of baseline and non-baseline segments.

f) Overhang Module 1512

In the specific example of implementation shown, the overhang module 1512 receives from the neighbourhood baseline module 1508 the signal indicative of the final set of baseline and non-baseline segments and the filtered fetal heart rate signal from low-pass filter 1500.

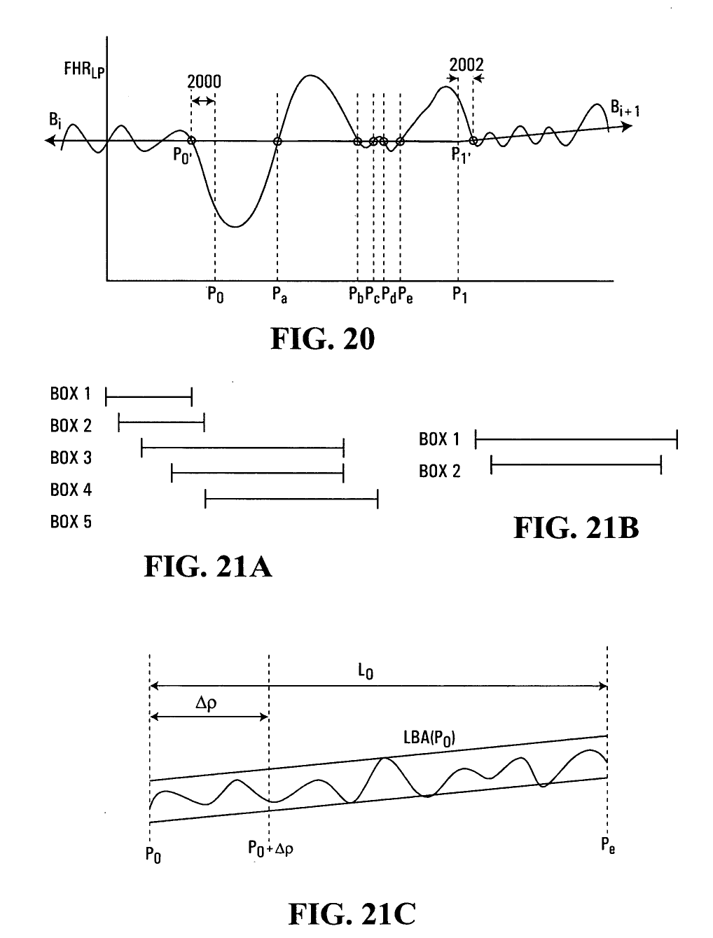

The overhang module 1512 allows extending the limits of segments classified as non-baseline segments to include transitions portions of the heart rate signal. This is best illustrated in FIG. 20 of the drawings. As shown, the initial estimation of the limits of baseline segments Bᵢ and Bᵢ₊₁ are P₀ and P₁ respectively. Therefore, the segments between P₀ and P₁ were classified as non-baseline segments. However, P₀′ and P₁′ are the intersections of the line of best fit of the baseline segment with the fetal heart rate signal. These intersection points are considered the extent possible of acceleration and deceleration events. In the specific example of implementation shown, overhang module 1512 is operative to clip the baseline segments that extend into acceleration segments and deceleration segments. In the example of FIG. 20, this is done by removing the portion of baseline segment Bᵢ between P₀′ and P₀, and by removing the portion of baseline segment Bᵢ₊₁ between P₁ and P₁′. Therefore the overhang module extends the boundary of the segments starting at P₀ to sample point P₀′ and the boundary of the segments ending at P₁ to sample point P₁′.

In addition, where line P₀′P₁′ intersects with the fetal heart rate signal (such as at points Pₐ through Pₑ) these intersection points are considered the extent of a possible acceleration or deceleration event.

g) Acceleration/Deceleration Module 1510

In the specific example of implementation shown, the acceleration/deceleration module 1510 receives from the neighbourhood baseline module 1508 the signal indicative of the final set of baseline and non-baseline segments and the filtered fetal heart rate signal from low-pass filter 1500.

With this information, the acceleration/deceleration module 1510 distinguishes the non-baseline segments as being either acceleration segments or deceleration segments. The procedure for processing the non-baseline segments can best be described with reference to FIG. 20.

The nature of the non-baseline segment is determined by processing the peak deviation between two points indicative of the extremities of non-baseline segments. If the peak deviation between two of these points is greater than a threshold, the segment is labelled acceleration or deceleration. For example, if the peak deviation between points P₀′ and Pₐ is below a certain deceleration threshold, this non-baseline segment would be classified as a deceleration segment. As a further example, if the deviation between points Pₐ and P_{b} is greater than a certain acceleration threshold, this non-baseline event would be classified as an acceleration segment. In yet a further example, if the peak deviation between intersection points P_{b} and Pₑ is neither greater than the certain acceleration threshold nor below the certain deceleration threshold then these non-baseline segments are unclassified or re-classified as baseline. In a specific implementation, the thresholds for determining acceleration and deceleration segments are linear functions of the local variability (calculated as the average FHRvar at P₀ and P₁).

In an alternative implementation, the thresholds for determining acceleration and deceleration segments are fixed values. Such fixed values may be established based on observations and/or experimental results.

### SECOND SPECIFIC EXAMPLE OF IMPLEMENTATION

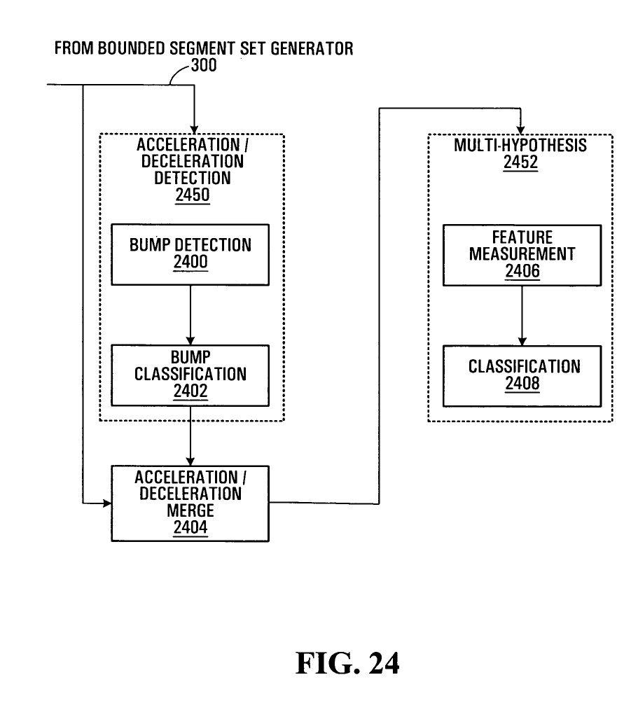

A feature identification unit 302 in accordance with a second specific example of implementation of the present invention is shown in FIG. 24. As shown, feature identification unit 302 receives the set of segments from bounded segment set generator 300 and the fetal heart rate from input 102. The feature identification unit 302 includes an acceleration/deceleration detection phase 2450, an acceleration/deceleration merge 2404 phase and a multi-hypothesis phase 2452. The acceleration/deceleration detection phase 2450 phase processed the set of segments received from bounded segment set generator 300 to detect candidate accelerations and decelerations. Following this, the acceleration/deceleration merge 2404 phase processes the candidate accelerations and decelerations as well as the set of segments received from bounded segment set generator 300 to further refine the labeling of accelerations and decelerations. The acceleration/deceleration merge 2404 phase releases a set of acceleration and deceleration sections and a set of candidate baseline sections. The multi-hypothesis phase 2452 processes the set of candidate baseline sections to remove section sections that do not fulfill certain baseline criteria. The multi-hypothesis phase 2452 releases a set of labelled acceleration sections, deceleration sections and baseline sections. Each of the above phases is described in greater detail below.

Acceleration/Deceleration Detection Phase 2450

Episodes of peaks and valleys in the foetal heart rate (FHR) signal are known as accelerations and decelerations, respectively. Their duration is typically in the range of 15 seconds to several minutes. In a non-limiting implementation, these events are detected in a two-step manner by first identifying candidate “bumps” (peaks or valleys) in the signal, and then classifying these candidates using a neural network. While detection of accelerations is done independently from that of decelerations, the symmetry of the two problems permits a common approach to be used. The following discussion applies to either acceleration or deceleration detection, unless distinctions between the two problems need to be noted. The acceleration/deceleration detection phase 2450 includes a bump detection step 2400 and a bump classification step 2402. Each of these step is described below in greater detail.

Bump Detection Step 2402

The first step is to collect a set of bumps in the signal that might correspond to accelerations or decelerations. This is done by first filtering the foetal heart rate signal to include only the frequency band of the feature events of interest, then performing peak detection to identify the feature event positions and finally using DC intersection to identify the feature event time extents.

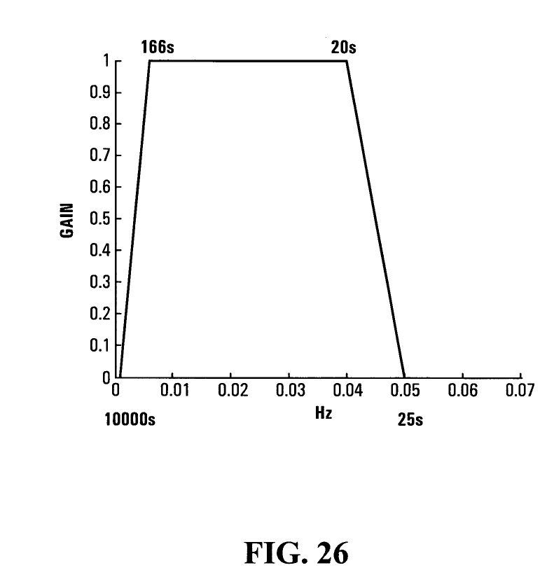

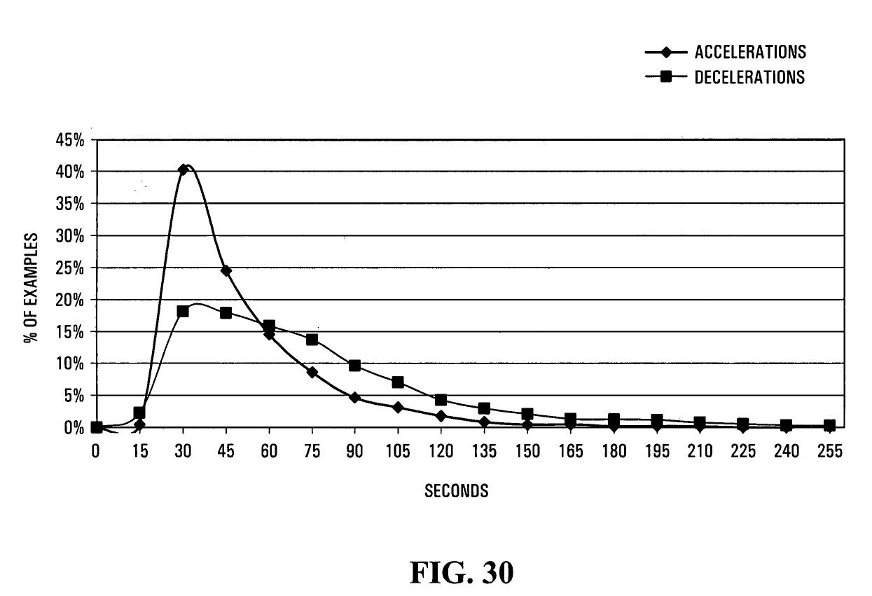

In a non-limiting implementation, the foetal heart rate is first filtered by a band-pass filter. In the obstetrical field, the generally agreed-upon minimum event duration for accelerations and deceleration is 15 second. Events can last up to approximately five minutes, although some decelerations can last even longer. Probability distributions for accelerations and deceleration are shown in FIG. 30. The probability distributions depicted is based on population sizes of 2540 and 5119 events for accelerations and decelerations, respectively, taken from 161 sample tracings. With this information a band-pass filter was created with the frequency response shown in FIG. 26. As depicted the cutoff frequencies correspond to 166 s (1/166 Hz) and 20 s (0.04 Hz), respectively. Zero-gain points (in reality, −70 dB gain) occur at 10000 s (1/10000 Hz) and 25 (0.05 Hz) s, respectively. In a practical implementation, the band-pass filter was implemented with a low-pass filter followed by a high-pass filter with the following specifications:

Low-pass filter

Rp=3 [dB], Fp=0.04 [Hz], Rs=70 [dB], Fs=0.05 [Hz], Order=750, Del=375

High-pass filter

Rp=3 [dB], Fp=0.006 [Hz], Rs=70 [dB], Fs=0.00001 [Hz], Order=1443, Del=771

Where Rp is the pass-band ripple, Fp is the cutoff (−3 dB) frequency, Rs is stop-band ripple, Fs is stop-band (−70 dB) frequency and Del is the group delay of the filter.

After this filtering is done, the high frequency content (bumps of short duration) and the low frequency component (containing the energy of the FHR offset) are removed from the signal.

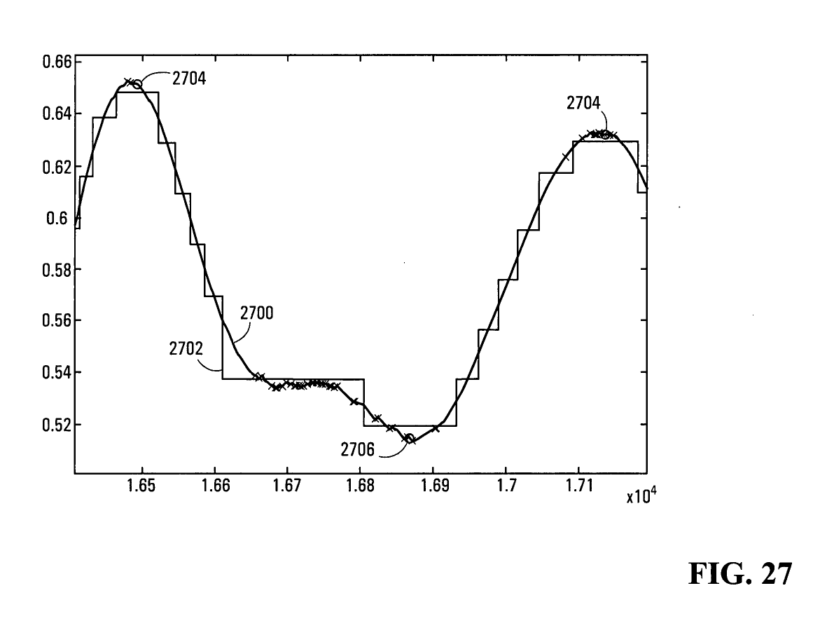

Following the filtering, the signal 2506 is processing by performing peak detection to identify the feature event positions. A straightforward method of detecting the peaks of the band-pass filtered signal is to approximate the first and second derivatives as difference and second-difference functions, respectively, and then detect their zero crossings. Due to quantization noise in the original signal however, this approach may detect many small insignificant peaks. To overcome this, in an alternative implementation, the signal 2506 is re-sampled using coarser quantization levels. Peaks are found when changes in the monotonicity of the quantized signal are found that exceed a certain threshold above the local average signal value. This threshold is chosen to be finer than the quantization resolution. This process is shown in FIG. 27 with sample values of quantization step size and height thresholds of 10⁻² and 10⁻⁴ bpm (beats per minute), respectively. As illustrated, to determine the peaks of the original FHR signal 2700 derivative zero-crossing techniques detect many insignificant peaks, shown as crosses is FIG. 27, due to quantization noise. To overcome this, the signal is resampled using coarser quantization 2702. Changes in the monotonicity of this signal are used to detect positive peaks 2704 and negative peaks 2706, shown as circles in FIG. 27.

Once the peaks have been detected, the positions of the candidate bumps are known. To determine the beginning and ending points of the feature event, a fixed-length window of the filtered signal, centered upon the peak, is extracted. The first DC (“direct current” or zero-frequency) intersections of the window, before and after the peak are determined by locating zero crossings of the filtered signal. These samples establish the time extent of the event. The fixed-length window is currently chosen to be 6 min 40 s long, sufficiently long enough to capture the vast majority of the events.

The low-frequency response of the band-pass filter allows some short duration or small area bumps to be passed and detected. These are removed using duration and area thresholds of 15 seconds and 4.17 beats respectively (the units of the area are “beats” considering the area product beats per unit time×time). In this non-limiting implementation, the area threshold is chosen to remove the smallest events while leaving further discrimination to subsequent steps. It is to be noted that it is a generally accepted obstetrical definition that accelerations and decelerations be at least 15 s long and 15 bpm in height. It is to be appreciated that other thresholds may be used without detracting from the spirit of the invention. Therefore an upper bound on the minimum-area threshold, assuming a (unrealistic but for simplicity purposes) square bump, would be 15 s×15 bpm×1 min/60 s=3.75 beats. In a practical implementation, a value slightly greater than 3.75 beats was used (determined by experimentation) to reduce the number of false events detected while maintaining the true-event sensitivity as high as possible.

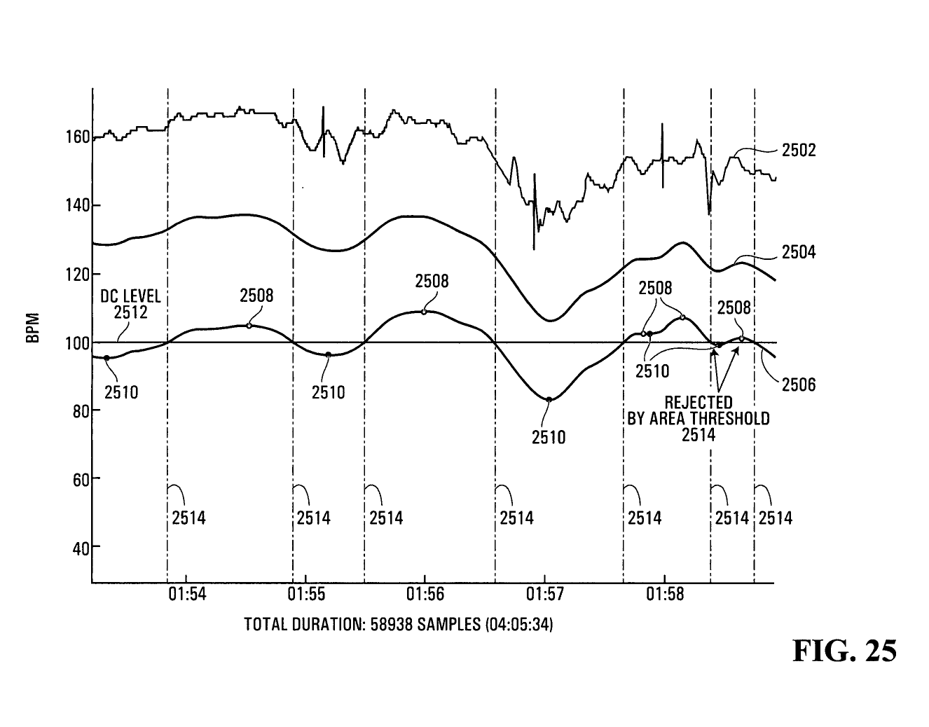

An example of the bump detection analysis step is shown in FIG. 25. As depicted, the original foetal heart rate signal 2502 is low-pass filtered to yield filtered signal 2504. The filtered signal 2504 is then high-pass filtered to yield signal 2506. The signal 2506 is then processed using any suitable known technique to detect positive and negative peaks. The positive peaks are labelled 2508 and the negative peaks are labelled 2510 in FIG. 25. The DC level 2512, normally at 0 beats per minute (BPM), but shifted up to 100 BPM for purposes of this diagram, intersects the signal at the dashed lines 2414. The dashed lines 2414 surrounding peaks delineate feature event time extents. Candidate accelerations and decelerations are indicated with ‘A’ and ‘D’ respectively. The rejections of a deceleration and an acceleration of insufficient areas are shown.

Bump Classification Step 2402

Given the set of candidate bumps determined at step 2402, there are still many false events to be removed. To accomplish this, more detailed measurements of the event shape and timing characteristics are performed. In a non-limiting implementation, a neural net is trained to discriminate true and false feature events in a representative data set using these measurements and expert markings. Given sufficient numbers of event examples during training, this network can be used to classify events not seen in the training event population.

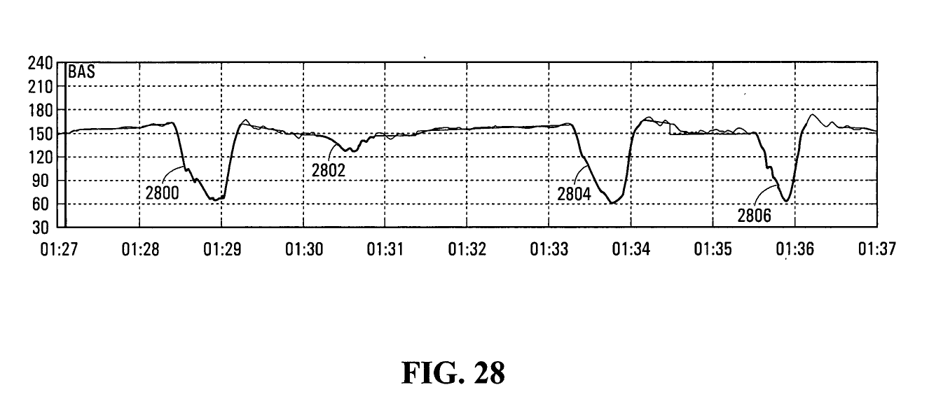

In a non-limiting implementation, a data set of 161 foetal heart rate cases that included both healthy and pathologic foetal heart rate patterns was used to build a neural network classifier. The first step was to create a gold standard using the manual markings of an expert obstetrician. In addition to marking the accelerations and deceleration, the decelerations were sub-classified into two groups according to their shape. These sub-classifications are known in the obstetrical field as abrupt (or ‘variable’) and gradual (or ‘late’) decelerations. Abrupt events tend to be deeper and of steep slope while gradual events are shallower and of lesser slope, and these characteristics have particular physiological significance. Examples of the two categories of decelerations in a sample FHR tracing are shown in FIG. 28. The first 2800, third 2804 and fourth negative peaks 2806 are examples of abrupt decelerations while the second 2802 is a gradual deceleration. From these markings, 2722 accelerations and 5831 decelerations (comprising abrupt and gradual events in roughly a 3:1 ratio) were identified.

The bump detection step 2400 (described above) was then independently performed on this population. Bumps were classified as true events if they overlapped with the expert markings by at least 25% and false otherwise. The overlap proportion was calculated using the longest event of the two in the denominator:

Overlap %=samples_{O}/max(samples_{E}, samples_{D})×100%

where

samples_{O} is the number of overlap samples

samples_{E} is the number of expert event samples

samples_{D} is the number of detected event samples

From this comparison, the bump-detection data is classified into a set of true and false feature events. In order to better characterize the feature event, a set of its measurements is taken. In a non-limiting implementation, this set includes:

Length: the time duration of the event

Onset: the time from the beginning to 90% of the peak value

Recovery: the time from 90% of the peak value to the end

FhrBegin: the FHR values at the beginning of the event

FhrEnd: the FHR values at the end of the event

FhrStd: the standard deviation of the FHR over the event

FhrInSlopeVal: the steepest slope during event onset

FhrInSlopeTime: the time from the beginning to FhrInSlopeVal

FhrOutSlopeVal: the steepest slope during event recovery

FhrOutSlopeTime: the time from the beginning to FhrOutSlopeVal

FhrPrev: the FHR level in the baseline immediately preceding event

FhrNext: the FHR level in the baseline immediately following event

Area: the sum of differences between the mean FHR and the FHR at each event sample

VarMax: the maximum variability over the event

VarPrev: the minimum variability in the baseline preceding the event

VarNext: the minimum variability in the baseline following the event

ContractionBegin: the time elapsed since the onset of the most recent contraction

ContractionEnd: the time elapsed since the end of the most recent contraction

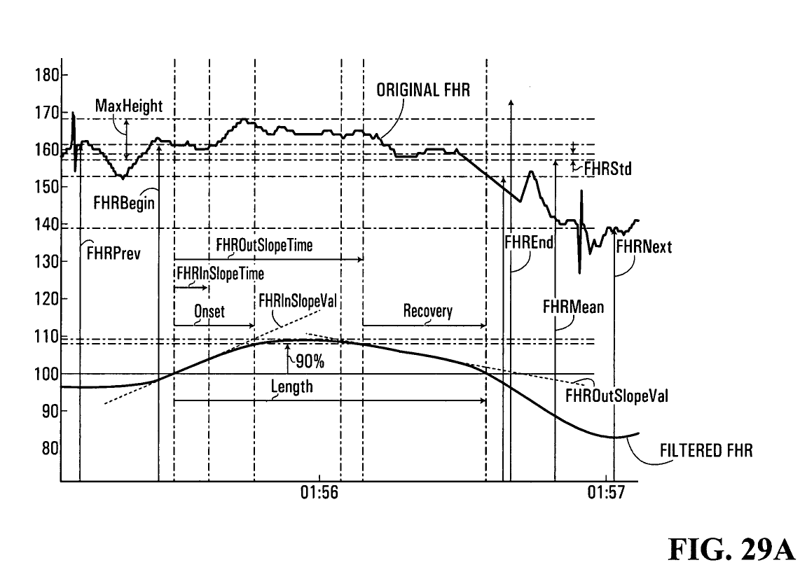

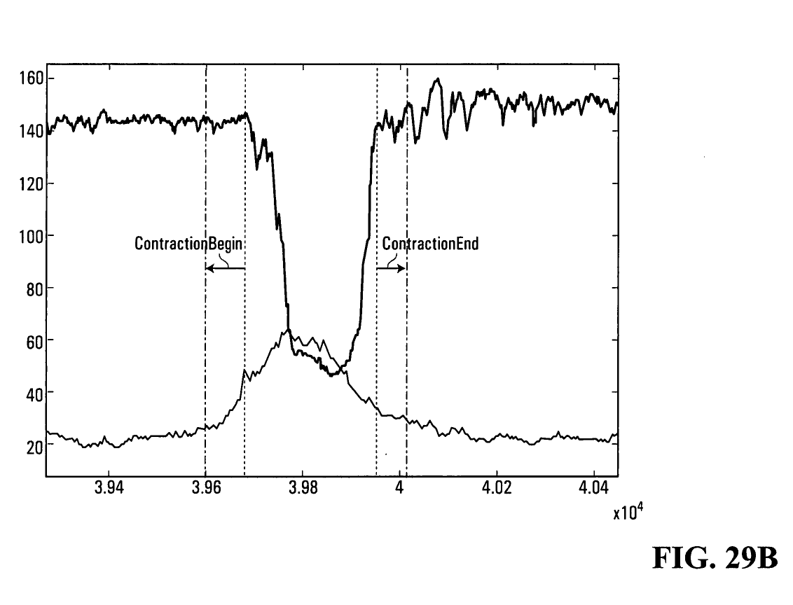

A diagram of these measurements for a candidate acceleration is shown in FIG. 29a and a candidate deceleration is shown in FIG. 29b.

Given these measurements of classified events, training of a standard feed-forward neural net is performed using the measurements as inputs and the classification as target outputs. For accelerations the possible target vectors are acceleration [1 0] or non-acceleration [0 1] (two outputs). For decelerations they are abrupt deceleration [1 0 0], gradual deceleration [0 1 0] and non-deceleration [0 0 1] (three outputs). Training was performed using Levenberg-Marquardt backpropagation, a standard second-order training algorithm. The current architecture of the network is 4×4×2 nodes for accelerations and 4×4×3 nodes for decelerations.

In this way, two neural networks are created: one for accelerations and another for decelerations. To assess the performance of the trained neural networks, an independent set of events was set aside during training. These events are classified and compared with the known expert classification. Assuming satisfactory training results, this same neural net is used in the production system to classify events in real-time. The bump classification step 2402 releases a set of accelerations acceleration sections and a set of deceleration sections.

Acceleration/Deceleration Merge Phase 2404

Some acceleration sections and deceleration sections detected at steps 2400 and 2402 may overlap with surrounding segments received from the bounded segment set generator 300. To avoid elaborate conflict resolution, we have chosen to favour decisions made on accelerations and decelerations over those made on baselines. The merge scheme is to first overwrite the segments received from the bounded segment set generator 300 with the set of acceleration segments, and then overwrite the result with the set of deceleration segments, removing any portions of the segments received from the bounded segment set generator 300 that overlap in each of the two steps.

The acceleration/deceleration merge 2404 phase releases a set of acceleration and deceleration sections. The portions of the segments received from the bounded segment set generator 300 and not labelled as acceleration and deceleration sections are released as a set of candidate baseline sections.

Multi-Hypothesis 2452

The multi-hypothesis phase 2452 processes the set of candidate baseline sections generated by the acceleration/deceleration merge 2404 phase and identifies baseline sections and non-baseline sections. This process is described below.

The multi-hypothesis phase 2452 identifies some baseline sequences that include sharp brief transitions where the baseline level is briefly displaced before returning to the original level. These transitions may be more accurately described as non-baseline and are thus removed from the baseline list. This phase addresses the same problem as the neighbourhood baseline module 1508 where the baseline may take on a few related levels and the correct baseline level must be determined (as in FIG. 18). The term “multi-hypothesis” refers to the problem of selecting the correct “hypothesis” of related baselines from a set of competing hypotheses. Distinguishing baselines from non-baselines is achieved by analyzing a window of a fixed number of consecutive baselines and presenting the resulting metrics to a neural network for classification. This again requires the creation of a gold standard and in this case the standard defines true baselines.

In order to train a neural network for the baseline detection phase 2452, the same set of 161 representative files was used to create the baseline gold standard. Again the expert obstetrician manually identified the baseline regions. Computer results from the baseline detection processing are compared to the gold standard to create a list of baselines labeled as either true or false. The comparison is the same overlap criterion used with acceleration and decelerations.

More specifically, for each baseline bᵢ of the n baselines in the window, the following feature measures are calculated:

Lengthᵢ: the length of bᵢ

YBeginᵢ: the FHR value at the beginning of the bᵢ

YEndᵢ: the FHR value at the end of bᵢ

YMeanᵢ: the mean FHR level for bᵢ

YStdᵢ: the standard deviation of the FHR level for bᵢ

Gapᵢ: the gap between bᵢ and bᵢ₊₁

GapMeanᵢ: the mean FHR in the gap between bᵢ and bᵢ₊₁

GapStdᵢ: the standard deviation of the FHR in the gap between bᵢ and bᵢ₊₁

The gap after bₙ is not considered. In the current implementation we use n=5 baselines for the size of the window.

The measurements performed on the n baselines are used to learn the classification of the j-th baseline. All the measures are provided to the input of a neural network and the target vector is either [0] (for baseline) or [1] for non-baseline, using the labels determined by comparison with the gold standard. In the current implementation, the neural network learns the classification of the middle baseline (j=3).

Training is again done with Levenberg-Marquardt backpropagation using an architecture of 10×8×2 nodes.

To test the performance of the neural network trained above, a portion of the files of baselines are not included in the training. These are presented to the trained neural network and the resulting classification is used to either keep (classification “true”) or reject (classification “false”) the baseline.

The baseline detection phase includes a feature measurement step 2406 and a classification step 2408.

In the feature measurement step 2406, for each candidate baseline bᵢ in the set of baseline candidates released by the acceleration/deceleration merge 2404 phase and in the window, the above described feature measures are calculated.

Following this data is presented to the trained neural network and the resulting classification is used to either keep (classification “true”) or reject (classification “false”) the baseline. The sections labelled as true baselines are labelled as baseline feature events. The sections labelled as false baselines are relabelled as “unclassified”. These unclassified sections may be processed by subsequent steps in order to label them as acceleration or declarations. Alternatively, these sections may remain unclassified.

The multi-hypothesis phase 2452 releases a set of labelled acceleration sections, deceleration sections and baseline sections.

Once feature identification unit 302 has processed the fetal heart rate signal sections of the fetal heart rate signal have been determined to be baseline sections, acceleration sections, or deceleration sections. Optionally, some of the sections are unclassified. Once all the sections have been determined, the feature identification unit 302 labels the portions of the fetal heart rate signal within the baseline sections as being sections of the fetal heart rate signal that are indicative of a baseline event. In addition, the feature identification unit 302 labels the portions of fetal heart rate signal within the acceleration and deceleration sections as being sections of the fetal heart rate signal that are indicative of an acceleration event or deceleration event, respectively.

The feature identification unit 302 then releases a signal indicative of the list of labeled heart rate of sections 304 to the post-processing module 202. The signal includes a plurality of data elements that are each associated with a respective section and include a label component, the label component is indicative of whether the section is an acceleration event, deceleration event or baseline event.

Post Processing Module 202

As mentioned earlier in the specification, the post processing module 202 is coupled to the feature detection module 200 and is adapted to receive the signal indicative of the list of labelled sections. The post-processing module is able to process the list of sections in order to produce meaningful data for the user.

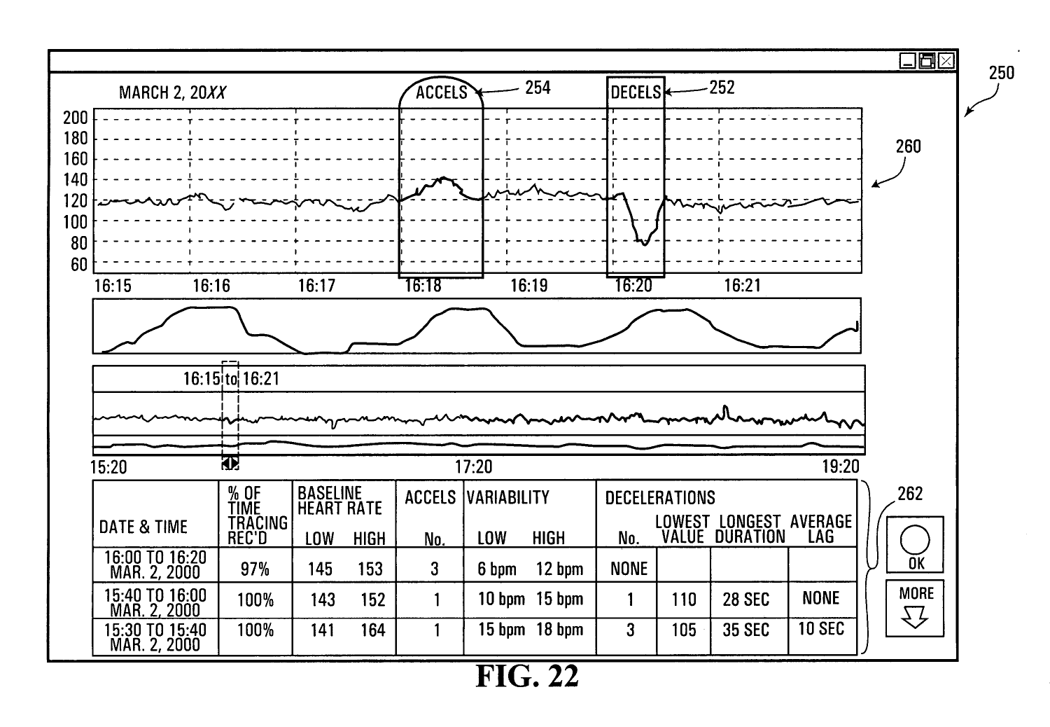

In a first non-limiting example, the post processing module 202 implements a graphical user interface for displaying heart rate information that can help the user to better asses the fetal heart rate. The information may be depicted in textual format, graphical format or any other suitable format for allowing the health care professionals to readily have access to the information. FIG. 22 shows a specific non-limiting examples of a graphical user interface 250 generated by post processing module 202. As shown the graphical user interface 250 includes a viewing window 260 for displaying at least an interval of the fetal heart rate signal. The viewing window may also include tabs 254 and 252 for highlighting acceleration and deceleration events. The graphical user interface 250 may also include a table portion 262 including meaningful information derived on the basis of the heart rate signal. The manner in which the information is derived and displayed is not material to the present invention and as such will not be described further here.

It will be readily appreciated that the post processing module 202 can be any device or apparatus that processes the list of labelled heart rate signal sections of in order to provide meaningful information to the user.

Specific Physical Implementation

Those skilled in the art should appreciate that in some embodiments of the invention, all or part of the functionality previously described herein with respect to the apparatus for segmenting a heart rate signal to identify heart rate feature events may be implemented as pre-programmed hardware or firmware elements (e.g., application specific integrated circuits (ASICs), electrically erasable programmable read-only memories (EEPROMs), etc.), or other related components.

In other embodiments of the invention, all or part of the functionality previously described herein with respect to the apparatus for segmenting a heart rate signal to identify heart rate feature events may be implemented as software consisting of a series of instructions for execution by a computing unit. The series of instructions could be stored on a medium which is fixed, tangible and readable directly by the computing unit, (e.g., removable diskette, CD-ROM, ROM, PROM, EPROM or fixed disk), or the instructions could be stored remotely but transmittable to the computing unit via a modem or other interface device (e.g., a communications adapter) connected to a network over a transmission medium. The transmission medium may be either a tangible medium (e.g., optical or analog communications lines) or a medium implemented using wireless techniques (e.g., microwave, infrared or other transmission schemes).

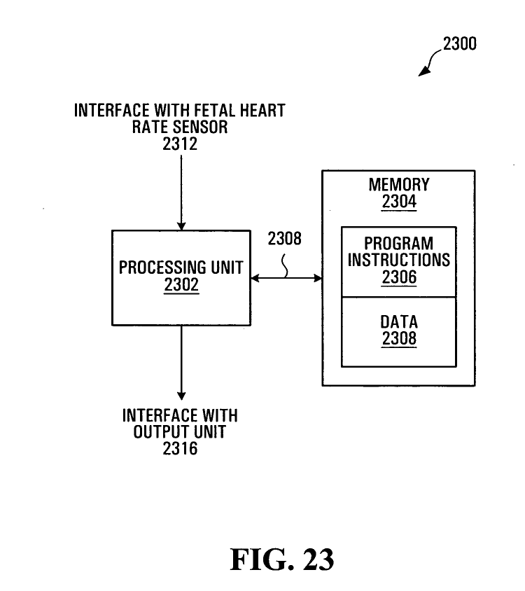

The computing unit implementing the apparatus for segmenting a heart rate signal to identify heart rate feature events may be configured as a computing unit 2300 of the type depicted in FIG. 23, including a processing unit 2302 and a memory 2304 connected by a communication bus. The memory includes data 2308 and program instructions 2306. The processing unit 2302 is adapted to process the data 2308 and the program instructions 2306 in order to implement the functional blocks described in the specification and depicted in the drawings. The computing unit 2300 may also comprise I/O interfaces 2312/2316 for receiving or sending data elements to external devices. For example, interface 2312 is for receiving a fetal heart rate signal and uterine activity signal.

Those skilled in the art should further appreciate that the program instructions 2306 may be written in a number of programming languages for use with many computer architectures or operating systems. For example, some embodiments may be implemented in a procedural programming language (e.g., “C”) or an object oriented programming language (e.g., “C++” or “JAVA”).

It will be appreciated that the system for monitoring the condition of a fetus may be of a distributed nature where the fetal heart rate signal is collected at one location by a fetal heart rate sensor and transmitted to a computing unit implementing the apparatus 100 over a network. The network may be any suitable network including but not limited to a global public network such as the Intranet, a private network and a wireless network. In addition, the computing unit implementing the apparatus 100 may be adapted to process multiple fetal heart rates originating from multiple fetuses concurrently using suitable methods known in the computer related arts.

Although the present invention has been described in considerable detail with reference to certain preferred embodiments thereof, variations and refinements are possible without departing from the spirit of the invention. Therefore, the scope of the invention should be limited only by the appended claims and their equivalents.

1. A method for segmenting a heart rate signal to identify heart rate feature events, said method comprising:
a) receiving a heart rate signal including a sequence of sample points;
b) processing the heart rate signal to generate a set of segments, each segment being formed by enclosing a portion of said heart rate signal in a respective bounded area, the bounded area commencing at a start sample point of said heart rate signal and terminating at an end sample point of said heart rate signal, wherein the sample points between said start sample point and end sample point lie within said bounded area;
c) processing the set of segments to generate a plurality of sections, each section being indicative of a heart rate feature;
d) releasing a signal indicative of said plurality of sections.

2. A method as defined in claim 1, wherein the heart rate feature is selected from the set consisting of an acceleration event, a deceleration event and a baseline event.

3. A method as defined in claim 1, wherein said bounded area is a trapezoid.

4. A method as defined in claim 3, wherein said trapezoid is a parallelogram.

5. A method as defined in claim 4, wherein for each segment, a polynomial approximation of the sample points between said start sample point and end sample point lies within the corresponding bounded area.

6. A method as defined in claim 5, wherein said polynomial approximation of the sample points between said start sample point and end sample point is a line of best fit.

7. A method as defined in claim 3, wherein a trapezoid associated with a given segment of said heart rate signal has a height conditioned at least in part on the basis of the variability of at least part of said heart rate signal.

8. A method as defined in claim 7, wherein the least part of said heart rate signal is the portion of the heart rate signal enclosed within said trapezoid.

9. A method as defined in claim 1, wherein said the bounded area is characterized by a certain drift and a certain excursion.

10. A method as defined in claim 9, wherein the certain excursion is conditioned at least on part on the basis of the heart rate signal.

11. A method as defined in claim 10, wherein the certain excursion is conditioned at least on part on the basis of a variability associated with the heart rate signal.

12. A method as defined in claim 3, wherein said signal indicative of said plurality of sections includes a list of labeled sections including a plurality of data elements, each data element being associated with a respective section and including a label component, the label component being indicative of either one of an acceleration event, deceleration event and baseline event.

13. A method as defined in claim 3, wherein said set of segments is generated using a recursive process.

14. A method as defined in claim 13, wherein said recursive process includes:
a) forming a segment of said set of segment by enclosing a portion of said heart rate signal in a bounded area, thereby leaving at least one remaining portion of the heart rate signal, the at least one remaining portion including sample points of the heart rate signal excluded from the enclosed portion;
b) repeating a) recursively for said at least one remaining portion of said heart rate signal until a certain condition is met.

15. A method as defined in 14, wherein the certain condition is met when the at least one remaining portion has a number of sample points below a pre-determined threshold number of sample points.

16. A method as defined in claim 1, where said heart rate signal is indicative of a fetal heart rate signal.

17. An apparatus for segmenting a heart rate signal to identify heart rate feature events, said apparatus comprising:
a) an input for receiving a heart rate signal including a sequence of sample points;
b) a first processing unit coupled to said input, said first processing unit being adapted for processing the heart rate signal to generate a set of segments, each segment being formed by enclosing a portion of said heart rate signal in a respective bounded area, the bounded area commencing at a start sample point of said heart rate signal and terminating at an end sample point of said heart rate signal, wherein the sample points between said start sample point and end sample point lie within said bounded area;
c) a second processing unit coupled to said first processing unit, said second processing unit being adapted for processing the set of segments to generate a plurality of sections, each section being indicative of a heart rate feature;
d) an output for releasing a signal indicative of said plurality of sections.

18. An apparatus as defined in claim 17, wherein the heart rate feature is selected from the set consisting of an acceleration event, a deceleration event and a baseline event.

19. An apparatus as defined in claim 17, wherein said bounded area is a trapezoid.

20. An apparatus as defined in claim 19, wherein said trapezoid is a parallelogram.

21. An apparatus as defined in claim 20, wherein for each segment, a polynomial approximation of the sample points between said start sample point and end sample point lies within the corresponding bounded area.

22. An apparatus as defined in claim 21, wherein said polynomial approximation of the sample points between said start sample point and end sample point is a line of best fit.

23. An apparatus as defined in claim 19, wherein a trapezoid associated with a given segment of said heart rate signal has a height conditioned at least in part on the basis of the variability of at least part of said heart rate signal.

24. An apparatus as defined in claim 23, wherein the least part of said heart rate signal is the portion of the heart rate signal enclosed within said trapezoid.

25. An apparatus as defined in claim 17, wherein said the bounded area is characterized by a certain drift and a certain excursion.

26. An apparatus as defined in claim 25, wherein the certain excursion is conditioned at least on part on the basis of the heart rate signal.

27. An apparatus as defined in claim 26, wherein the certain excursion is conditioned at least on part on the basis of a variability associated with the heart rate signal.

28. An apparatus as defined in claim 19, wherein said signal indicative of said plurality of sections includes a plurality of data elements, each data element being associated with a respective section and including a label component, the label component being indicative of either one of an acceleration event, deceleration event and baseline event.

29. An apparatus as defined in claim 19, wherein said first processing unit implements a recursive process for generating said set of segments.

30. An apparatus as defined in claim 29, wherein said recursive process includes:
a) forming a segment of said set of segment by enclosing a portion of said heart rate signal in a bounded area, thereby leaving at least one remaining portion of the heart rate signal, the at least one remaining portion including sample points of the heart rate signal excluded from the enclosed portion;
b) repeating a) recursively for said at least one remaining portion of said heart rate signal until a certain condition is met.

31. An apparatus as defined in claim 30, wherein the certain condition is met when the at least one remaining portion has a number of sample points below a pre-determined threshold number of sample points.

32. An apparatus as defined in claim 18, where said heart rate signal is indicative of a fetal heart rate signal.

33. A computer readable storage medium including a program element suitable for execution by a computing apparatus for segmenting a heart rate signal to identify heart rate feature events, said computing apparatus comprising:
a) a memory unit;
b) a processor operatively connected to said memory unit, said program element when executing on said processor being operative for:
i. receiving a heart rate signal including a sequence of sample points;
ii. processing the heart rate signal to generate a set of segments, each segment being generated by enclosing a portion of said heart rate signal in a respective bounded area, the bounded area commencing at a start sample point of said heart rate signal and terminating at an end sample point of said heart rate signal, wherein the sample points between said start sample point and end sample point lie within said bounded area;
iii. processing the set of segments to generate a plurality of sections, each section being indicative of a heart rate feature;
iv. releasing a signal indicative of said plurality of sections.

34. A computer readable storage medium as defined in claim 33, wherein the heart rate feature is selected from the set consisting of an acceleration event, a deceleration event and a baseline event 35. A computer readable storage medium as defined in claim 33, wherein said bounded area is a trapezoid.

36. A computer readable storage medium as defined in claim 35, wherein said trapezoid is a parallelogram.

37. A computer readable storage medium as defined in claim 36, wherein for each segment, a polynomial approximation of the sample points between said start sample point and end sample point lies within the corresponding bounded area.

38. A computer readable storage medium as defined in claim 37, wherein said polynomial approximation of the sample points between said start sample point and end sample point is a line of best fit.

39. A computer readable storage medium as defined in claim 35, wherein a trapezoid associated with a given segment of said heart rate signal has a height conditioned at least in part on the basis of the variability of at least part of said heart rate signal.

40. A computer readable storage medium as defined in claim 39, wherein the least part of said heart rate signal is the portion of the heart rate signal enclosed within said trapezoid.

41. A computer readable storage medium as defined in claim 34, wherein the bounded area is characterized by a certain drift and a certain excursion.

42. A computer readable storage medium as defined in claim 41, wherein the certain excursion is conditioned at least on part on the basis of the heart rate signal.

43. A computer readable storage medium as defined in claim 42, wherein the certain excursion is conditioned at least on part on the basis of a variability associated with the heart rate signal.

44. A computer readable storage medium as defined in claim 35, wherein said signal indicative of said plurality of sections includes a plurality of data elements, each data element being associated with a respective section and including a label component, the label component being indicative of either one of an acceleration event, deceleration event and baseline event.

45. A computer readable storage medium as defined in claim 35, wherein said program element implements a recursive process for generated the set of segments.

46. A computer readable storage medium as defined in claim 45, wherein said recursive process includes:
a) forming a segment of said set of segment by enclosing a portion of said heart rate signal in a bounded area, thereby leaving at least one remaining portion of the heart rate signal, the at least one remaining portion including sample points of the heart rate signal excluded from the enclosed portion;
b) repeating a) recursively for said at least one remaining portion of said heart rate signal until a certain condition is met.

47. A computer readable storage medium as defined in 46, wherein the certain condition is met when the at least one remaining portion has a number of sample points below a pre-determined threshold number of sample points.

48. A computer readable storage medium as defined in claim 34, where said heart rate signal is indicative of a fetal heart rate signal.

49. A fetal monitoring system comprising:
a) a sensor for receiving a signal indicative of a fetal heart rate;
b) an apparatus suitable for monitoring the condition of a fetus, said apparatus comprising:
i. an input coupled to said sensor for receiving a signal indicative of a fetal heart rate;
ii. a feature detection module coupled to said input, said feature detection module implementing:
(a) a first processing unit adapted for processing the heart rate signal to generate a set of segments, each segment being generated by enclosing a portion of said heart rate signal in a respective bounded area, the bounded area commencing at a start sample point of said heart rate signal and terminating at an end sample point of said heart rate signal, wherein the sample points between said start sample point and end sample point lie within said bounded area;
(b) a second processing unit adapted for processing the set of segments to generate a plurality of sections, each section being indicative of a heart rate feature;
iii. a post processing module coupled to said a feature detection module, said post processing module being adapted for deriving information on the basis of the heart rate features associated with said set of segments;
iv. an output for releasing the information derived from the heart rate features associated set of segments;
c) an output unit coupled to the output for said apparatus, said output unit being suitable for displaying the information derived from the heart rate features associated with said set of segments.

50. An apparatus for segmenting a heart rate signal to identify heart rate feature events, said apparatus comprising:
a) means for receiving a heart rate signal including a sequence of sample points;
b) means for processing the heart rate signal to generate a set of segments, each segment being formed by enclosing a portion of said heart rate signal in a respective bounded area, the bounded area commencing at a start sample point of said heart rate signal and terminating at an end sample point of said heart rate signal, wherein the sample points between said start sample point and end sample point lie within said bounded area;
c) means for processing the set of segments to generate a plurality of sections, each section being indicative of a heart rate feature;
d) means for releasing a signal indicative of said plurality of sections.
